(19) United States
(12) Patent Application Publication
(10) Pub. No.: US 2010/0190994 A1
(43) Pub. Date: Jul. 29, 2010

(54) THIAZOLE SYSTEM ORGANIC ELECTROLUMINESCENT COMPOUNDS AND ORGANIC LIGHT EMITTING DIODE USING THE SAME

(76) Inventors: Mi Ae Lee, Gyeonggi-do (KR); Hyuck Joo Kwon, Seoul (KR); Bong Ok Kim, Seoul (KR); Sung Min Kim, Seoul (KR); Seung Soo Yoon, Seoul (KR)

Correspondence Address: ROHM AND HAAS ELECTRONIC MATERIALS LLC, 455 FOREST STREET, MARLBOROUGH, MA, 01752, US

(21) Appl. No.: 12/450,487

(22) PCT Filed: Mar. 25, 2008
(86) PCT No.: PCT/KR2008/001659

§ 371 Date: Mar. 17, 2010

(30) Foreign Application Priority Data
Mar. 28, 2007 (KR) 10-2007-0030315

(51) Int. Cl. C07F 7/02; C07D 277/62

(52) U.S. Cl. 548/110; 548/156; 548/160

(57) ABSTRACT

The present invention relates to novel thiazole system organic electroluminescent compounds and organic light emitting diodes comprising the same. Since the thiazole system organic electroluminescent compounds according to the invention have good luminous efficiency and life property, OLED's having very good operation lifetime can be produced.

### TECHNICAL FIELD

The present invention relates to novel thiazole system organic electroluminescent compounds and organic light emitting diodes comprising the same.

### BACKGROUND ART

As the modern society comes into information-oriented age, the importance of a display, which plays a role of interface between the electronic information device and human being, increases. As a novel planar display technique, OLED's have been actively investigated all over the world, since OLED's show excellent display property as self-luminescent device, and the manufacture is easy because of simple device structure, and enable manufacturing of ultra-thin and ultra-light weight displays.

OLED device usually consists of a plurality of thin layers of organic compound between the cathode and anode made of metal. Electrons and holes injected through the cathode and anode are transmitted to an electroluminescent layer via an electron injection layer and an electron transportation layer, a hole injection layer and a hole transportation layer to form excitons, which degrade into stable state to emit light. In particular, the properties of an OLED largely depend on the properties of the organic electroluminescent compound employed. Accordingly studies on core organic materials having enhanced performances have been actively achieved.

The core organic materials are classified into electroluminescent materials, carrier injection and transportation materials. The electroluminescent materials can be classified into host materials and dopant materials. Usually, as the device structure with most excellent EL properties, structures comprising a core organic thin film layer employing host-dopant doping system have been known.

Recently, small size displays are practically used, so that development of OLED's with high efficiency and long life is raising as an urgent subject. This would be an important milestone in the field of practical use of medium to large size OLED panels. Thus, development of core organic materials having more excellent properties as compared to conventional core organic materials is urgently required. From this point of view, development of host materials, carrier injection and transportation materials is one of the important subjects to be solved.

Desirable properties for host material as solid state solvent and energy deliverer or material for carrier injection or delivery in an OLED are high purity and appropriate molecular weight to enable vacuum vapor deposition. In addition, they should ensure thermal stability with high glass transition temperature and thermal decomposition temperature, and they should have high electrochemical stability for long life of the product, and easily form an amorphous thin layer. Particularly, it is very important for them to have good adhesion with the material of other adjacent layers, along with difficulties in interlayer migration.

Representatives for conventional electron delivery material include aluminum complexes such as tris(8-hydroxyquinoline)aluminum (III) (Alq), which has been used prior to the multilayer thin film OLED's disclosed by Kodak in 1987; and beryllium complexes such as bis(10-hydroxybenzo-[h]quinolinato)beryllium (Bebq), which was reported in the middle of 1990's in Japan [T. Sato et al., J. Mater. Chem. 10 (2000) 1151]. However, the limitation of the materials has come to the fore as OLED's have been practically used after 2002. Thereafter, many electron delivery materials of high performance have been investigated and reported to approach their practical use.

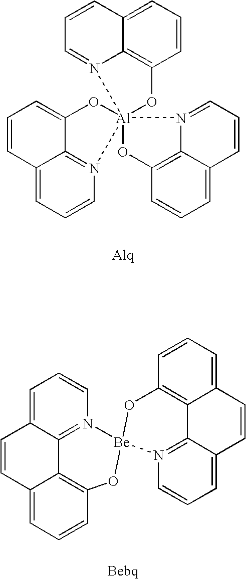

In the meanwhile, non-metal complex election delivery materials of good features which have been reported up to the present include spiro-PBD [N. Jahansson et al. Adv. Mater. 10 (1998) 1136], PyPySPyPy [M. Uchida et al. Chem. Mater. 13 (2001) 2680] and TPBI [Y.-T. Tao et al. Appl. Phys. Lett. 77 (2000) 1575] of Kodak. However, there remain various needs for improvement in terms of electroluminescent properties and lifetime.

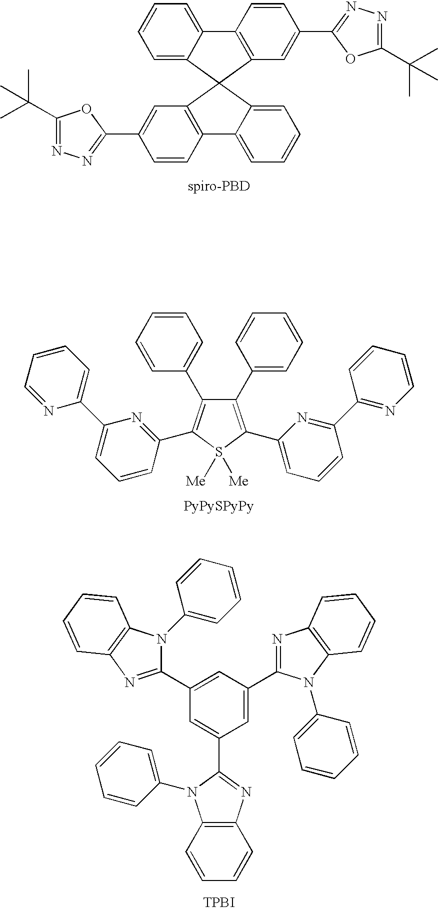

Particularly noticeable is that conventional electron delivery materials have only slightly improved operation voltage as compared to what was reported, or show the problem of considerable reduction of device operation lifetime. In addition the materials exhibit adverse effects such as deviation in device lifetime for each color and deterioration of thermal stability. Up to the present, those adverse effects are in the way to achieve the objects such as reasonable power consumption and increased luminance, which have been the issues in manufacturing large-size OLED panels.

### DISCLOSURE

### Technical Problem

The object of the present invention is to solve the problems described above, and to provide thiazole system organic electroluminescent compounds with improved electroluminescent properties, excellent power efficiency property and operation lifetime, as compared to that from conventional electron transportation materials. Another object of the present invention is to provide organic light emitting diode comprising said thiazole system organic electroluminescent compound.

### Technical Solution

The present invention relates to thiazole system organic electroluminescent compounds represented by Chemical Formula (1) and organic light emitting diodes comprising the same. Since the thiazole system organic electrolumescent compounds according to the present invention have excellent luminous efficiency, power efficiency and life property, OLED's having very good operation lifetime can be produced.

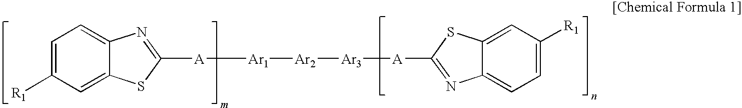

wherein, A is a chemical bond or

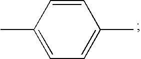

if m is 0, Ar₁ is hydrogen, phenyl, 1-naphthyl or 2-naphthyl;

if m is 1 or 2, Ar₁ is selected from following structures;

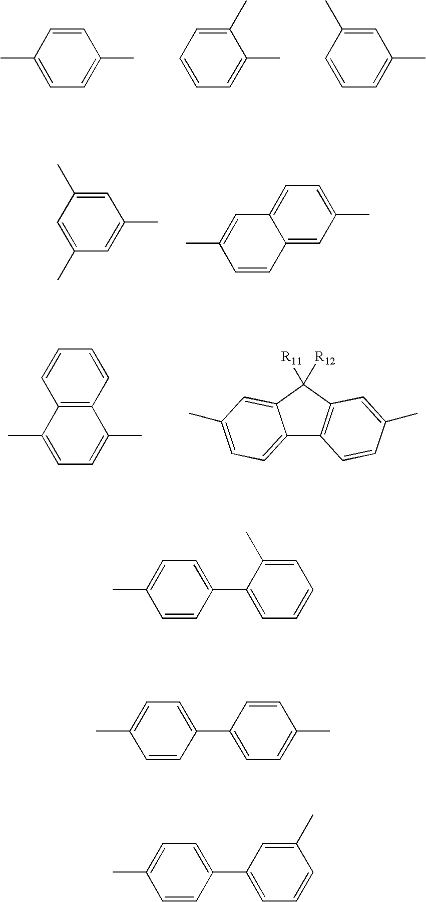

Ar₂ is selected from following structures;

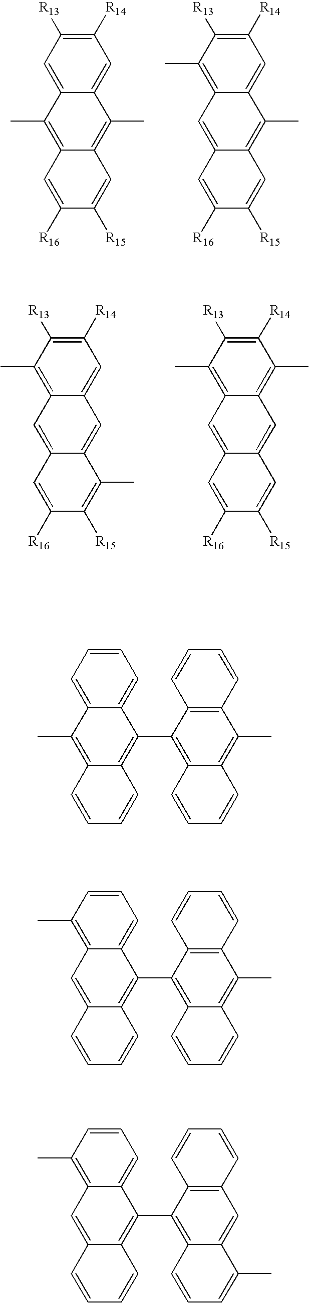

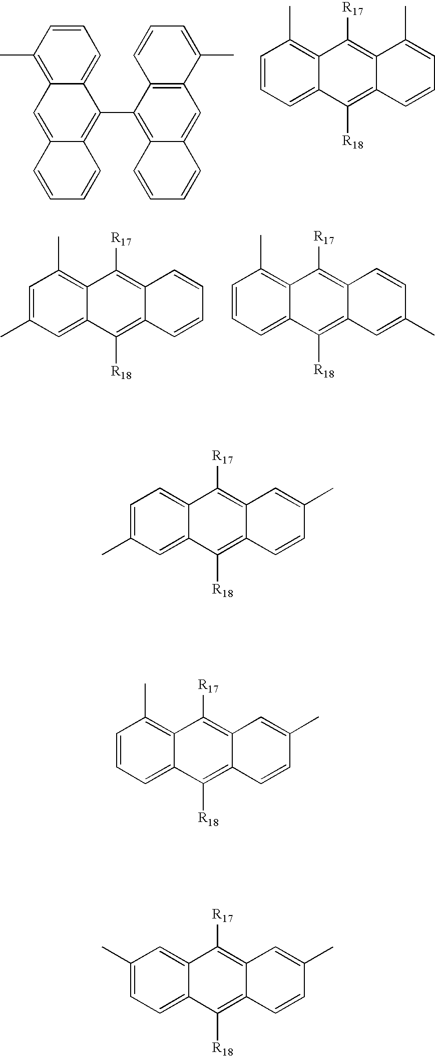

Ar₃ is selected from following structures;

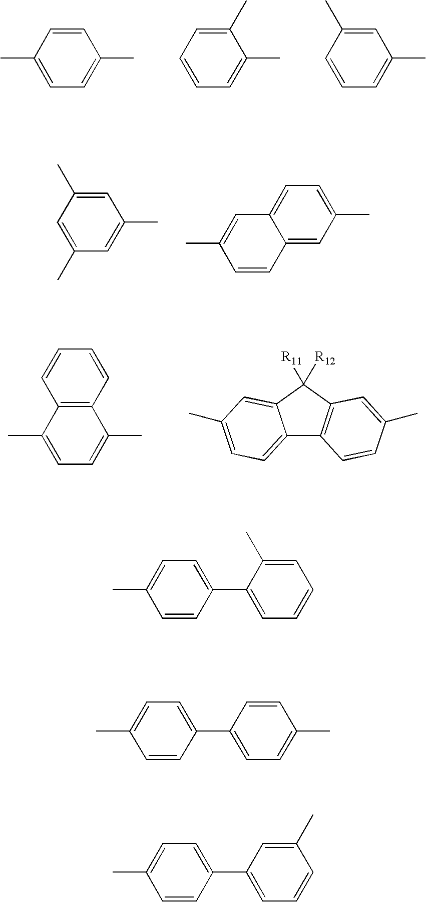

R₁ independently represents hydrogen, a C₁₋₂₀ alkyl group with or without halogen substituent(s), a C₁₋₂₀ alkylsilyl group, a C₆₋₂₀ arylsilyl group or a C₈₋₂₀ aryl group;

R₁₁ and R₁₂ independently represent hydrogen, or a C₁₋₂₀ alkyl group with or without halogen substituent(s);

R₁₃ through R₁₈ independently represent hydrogen, a C₁₋₂₀ alkyl group with or without halogen substituent(s), a C₁₋₂₀ alkylsilyl group, a C₈₋₂₀ arylsilyl group or a C₆₋₂₀ aryl group;

n is 1 or 2; and

the aryl group of R₁ and R₁₃ through R₁₈ may further comprise C₁₋₂₀ alkyl group(s) or halogen substituent(s).

If there is no element in A of the Chemical Formulas of the present invention, but Ar₁ or Ar₃ is simply bonded to the carbon of 2-position of thiazole is referred to as ‘a chemical bond’.

The thiazole system organic electroluminescent compounds of Chemical Formula (1) according to the present invention are exemplified by compounds of Chemical Formulas (2) to (4):

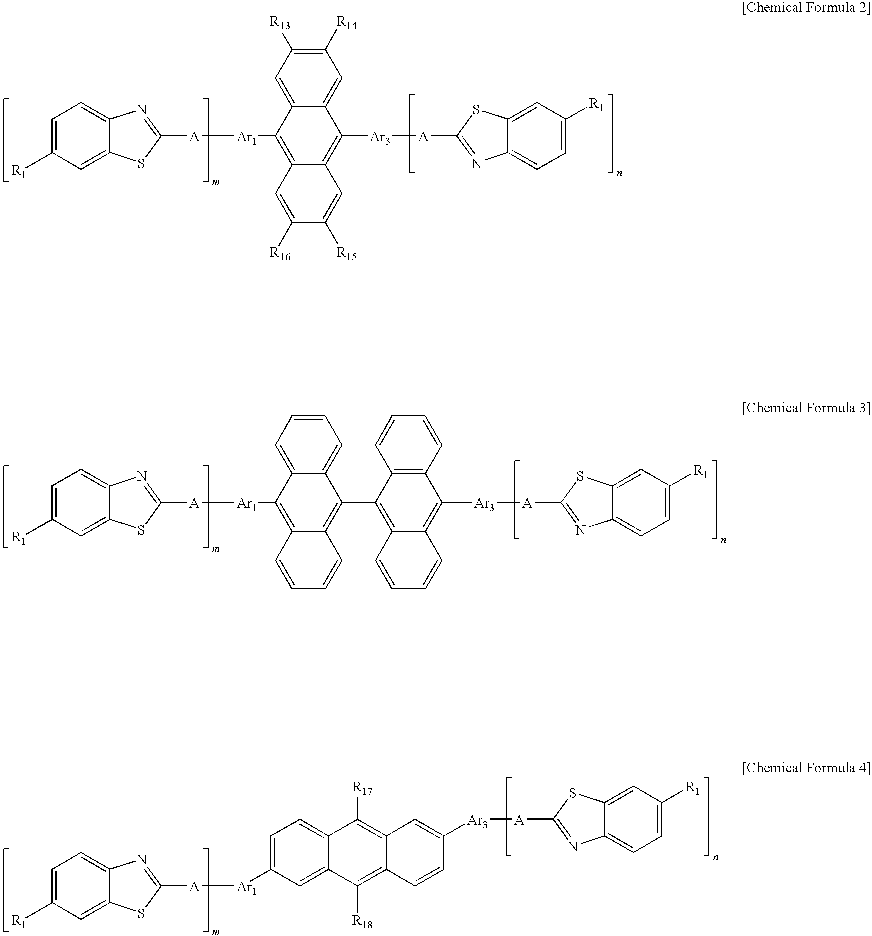

In Chemical Formulas (2) to (4), A, Ar₁, Ar₃, R₁, R₁₃, R₁₄, R₁₅, R₁₆, R₁₇, R₁₈, m and n are defined as for Chemical Formula (1).

In Chemical Formulas (1) to (4), R₁ and R₁₃ through R₁₅ are independently selected from hydrogen, methyl, ethyl, n-propyl, i-propyl, i-butyl, t-butyl, n-pentyl, i-amyl, n-hexyl, n-heptyl, n-octyl, 2-ethylhexyl, n-nonyl, decyl, dodecyl, hexadecyl, trifluoromethyl, pentafluoroethyl, trimethylsilyl, tripropylsilyl, tri(t-butyl)silyl, t-butyldimethylsilyl, triphenylsilyl, phenyldimethylsilyl, phenyl, benzyl, tolyl, 2-fluorophenyl, 4-fluorophenyl, biphenyl, naphthyl, anthryl, phenanthryl, naphthacenyl, fluorenyl, 9,9-dimethylfluoren-2-yl, pyrenyl, phenylenyl and fluoranthenyl.

The thiazole system organic electroluminescent compounds according to the present invention may be specifically exemplified by following compounds, but not restricted thereto.

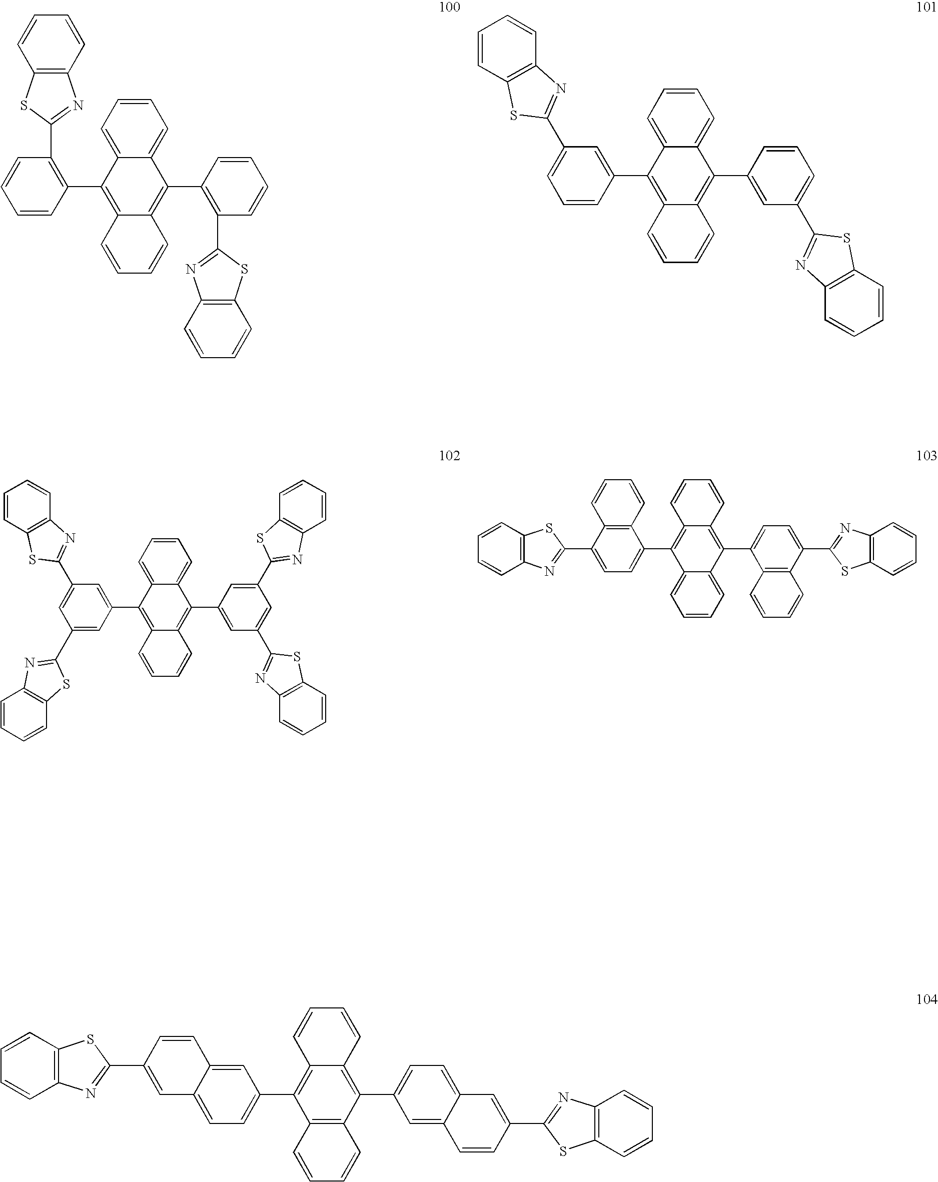

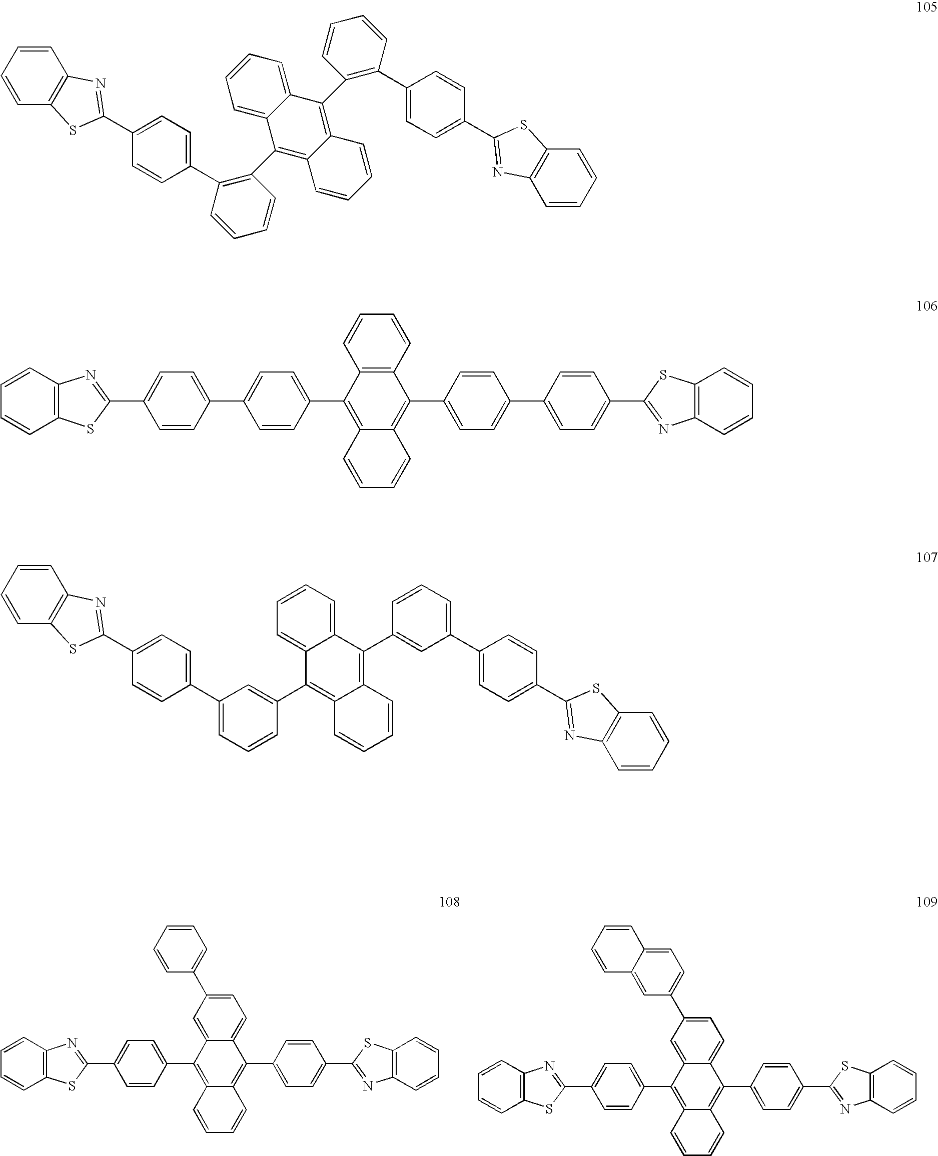

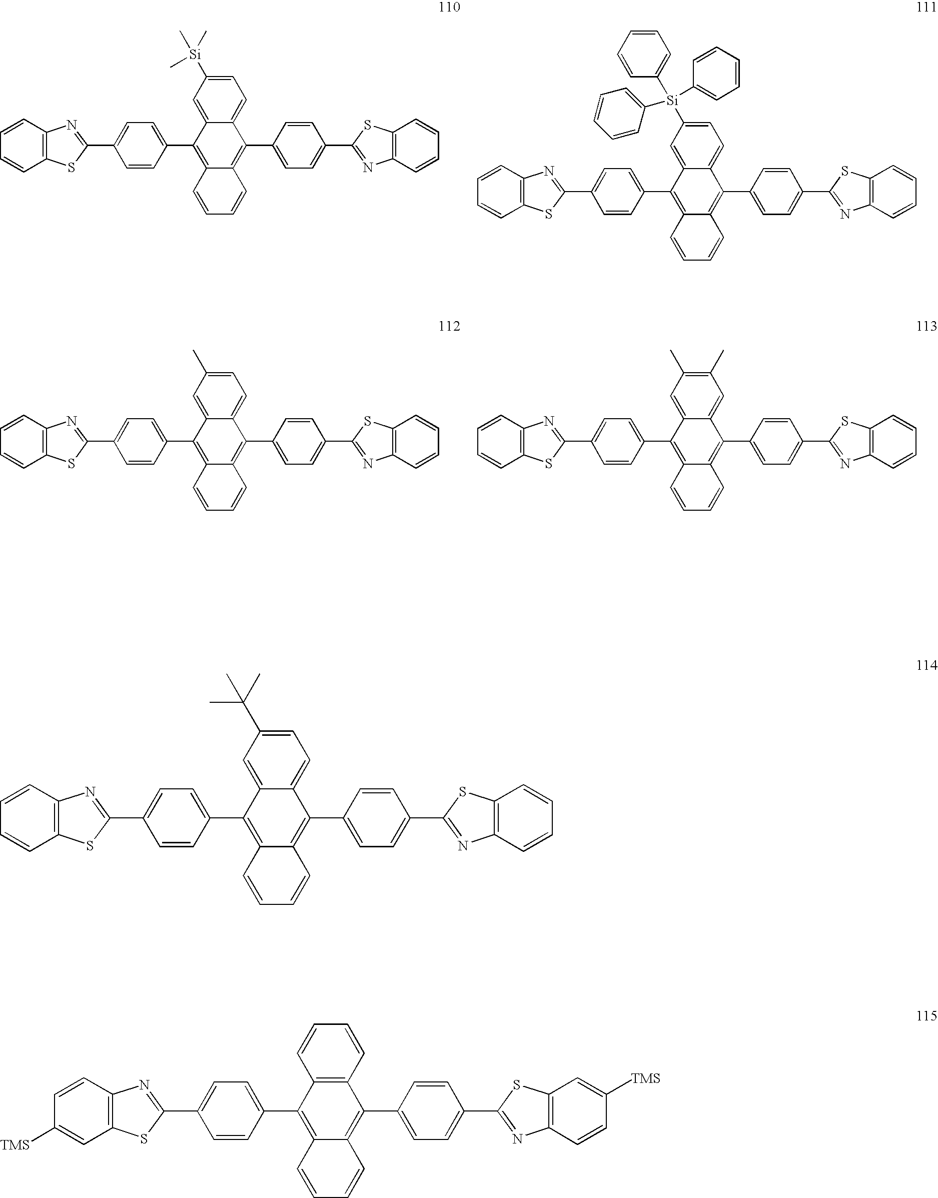

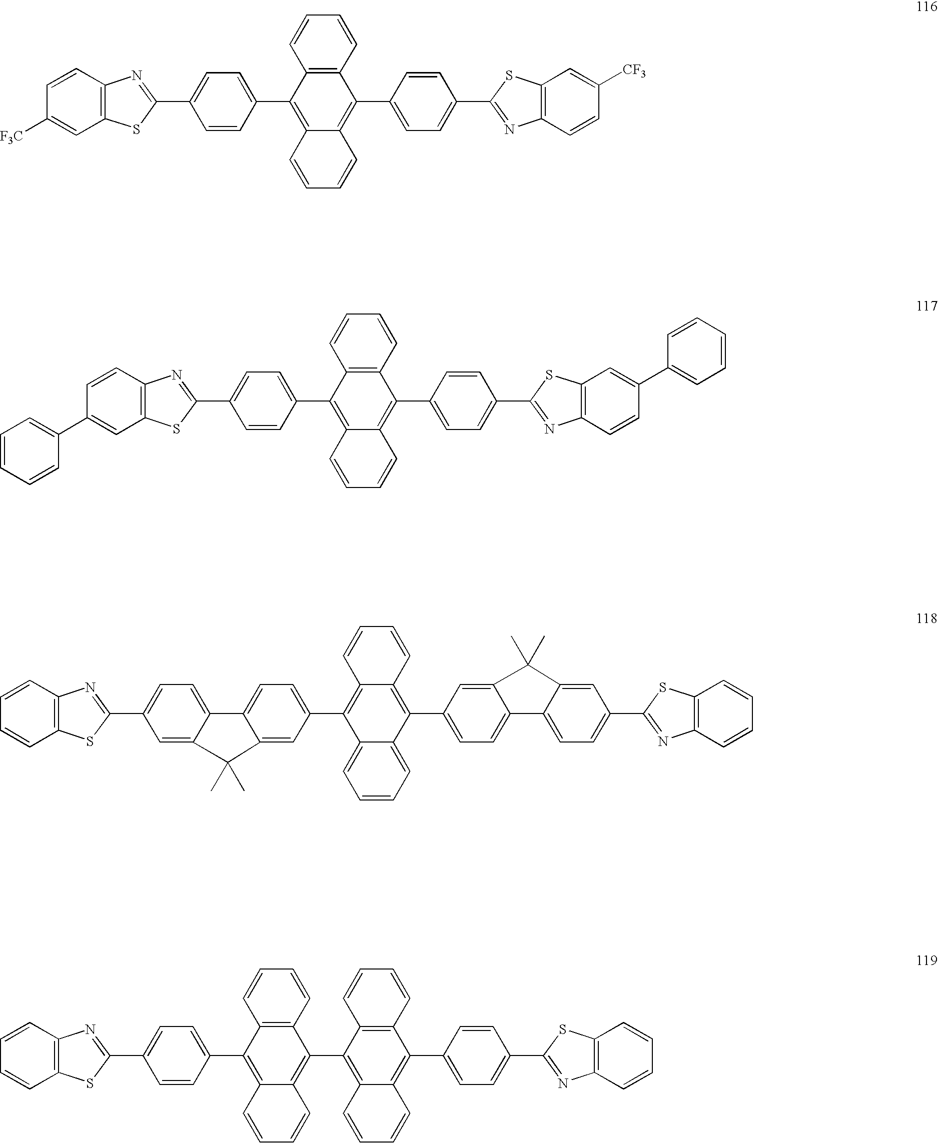

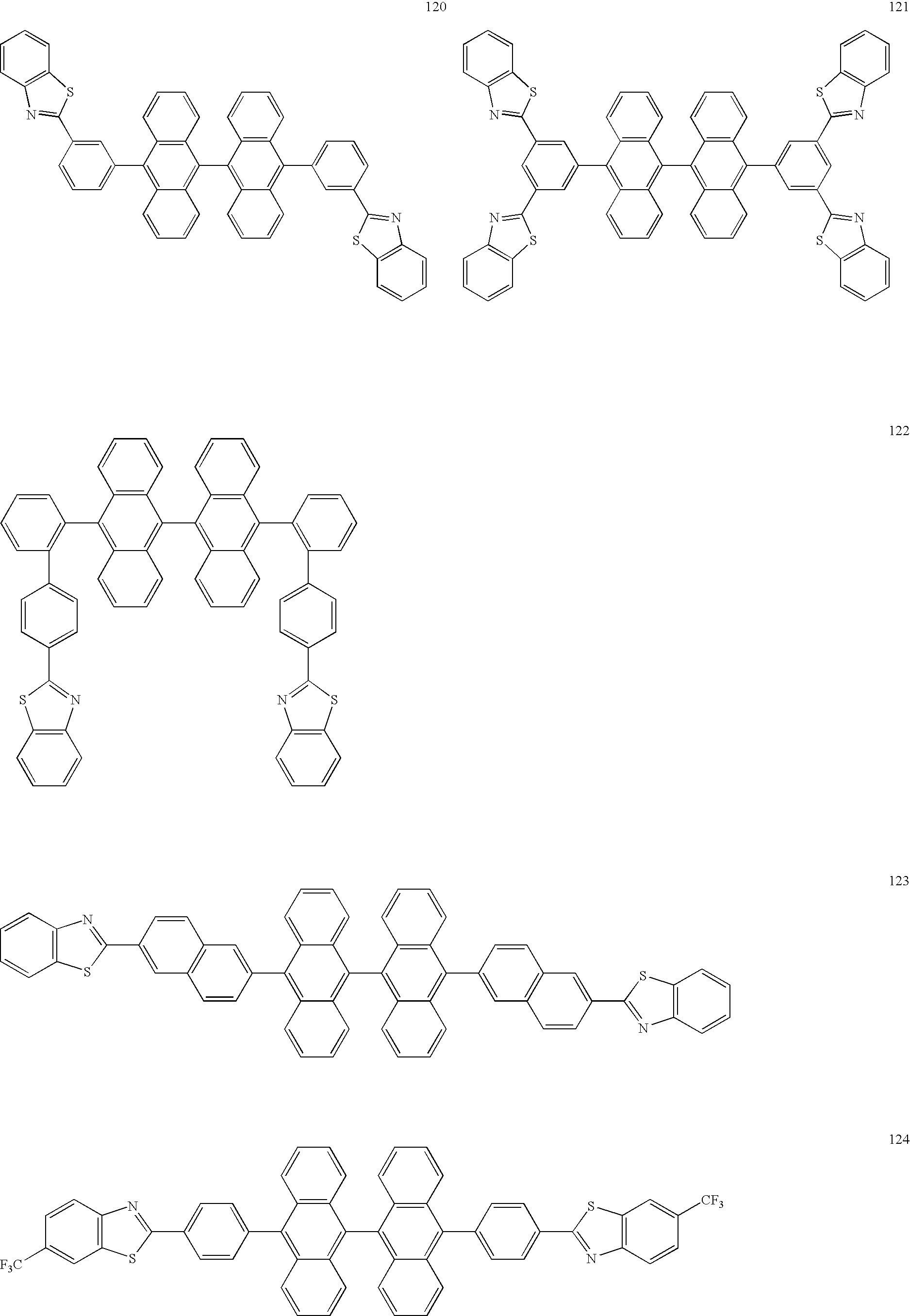

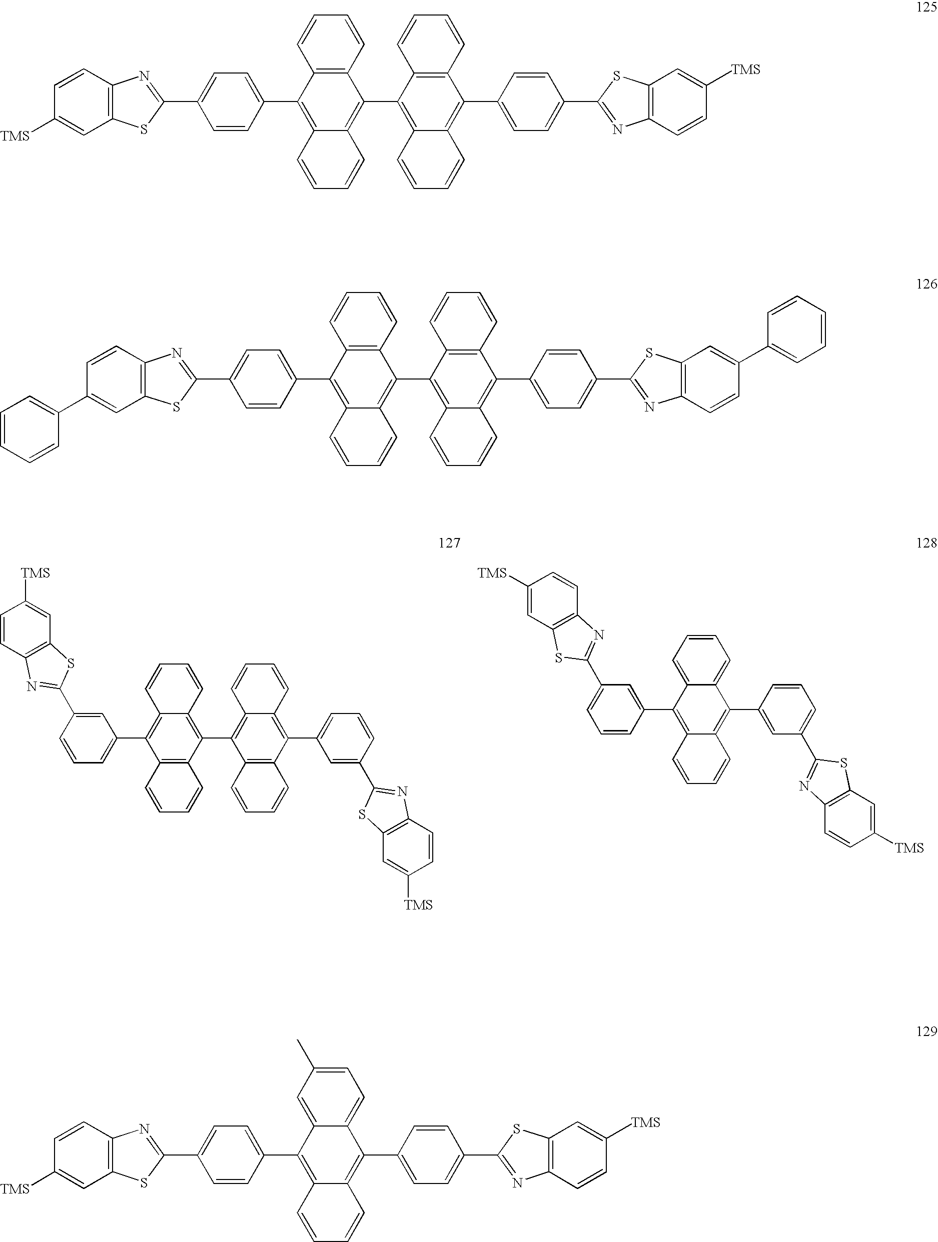

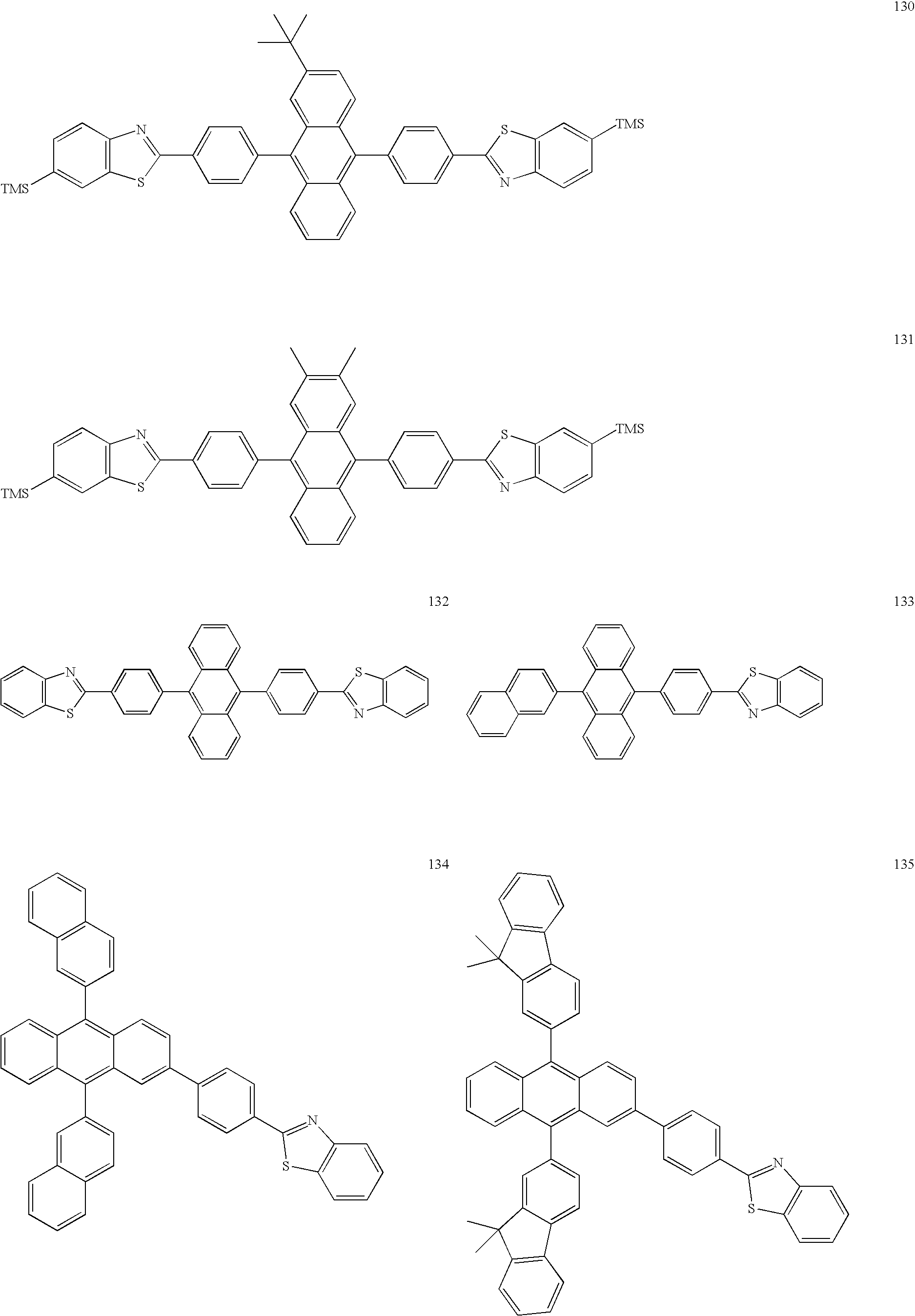

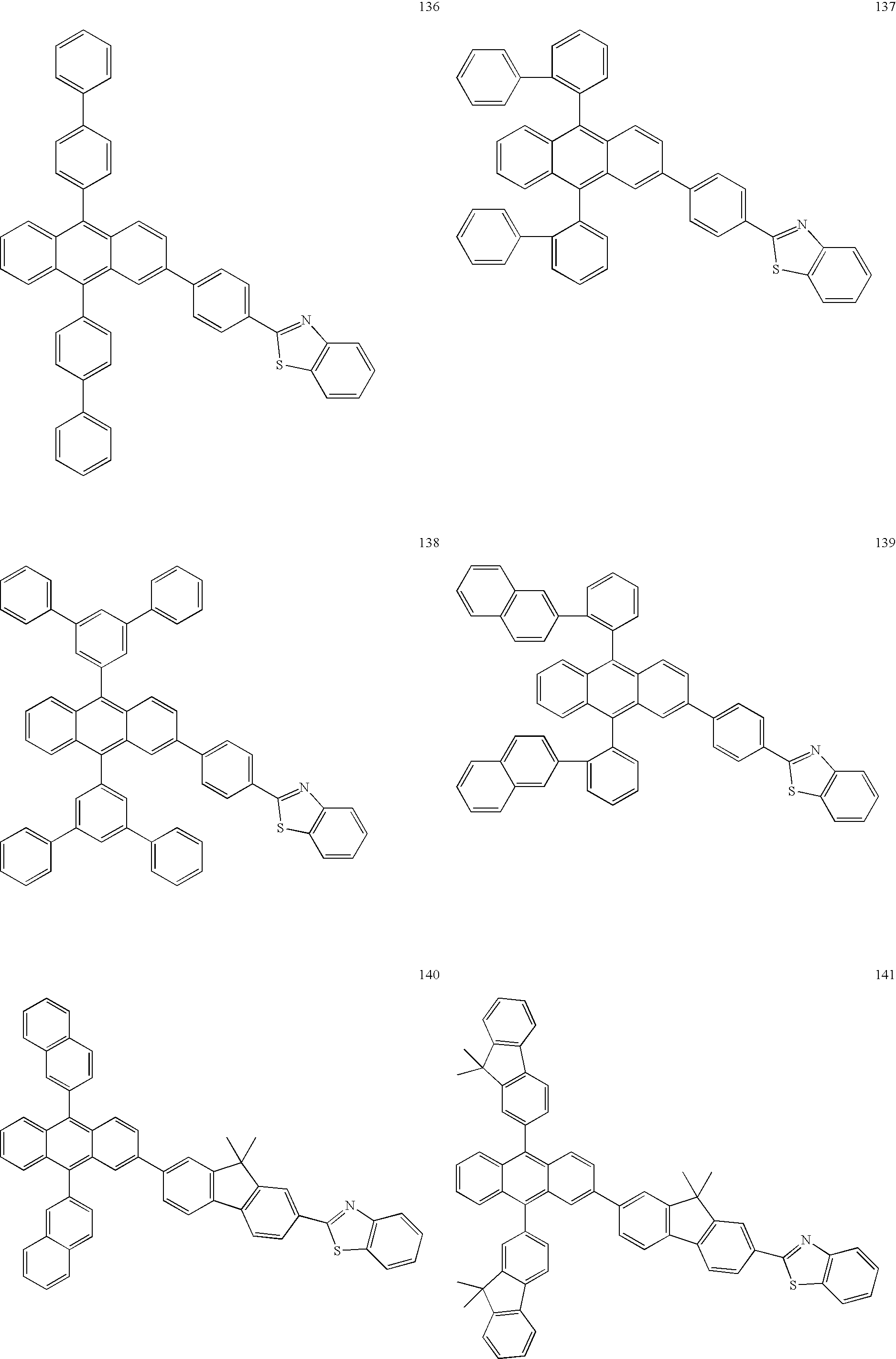

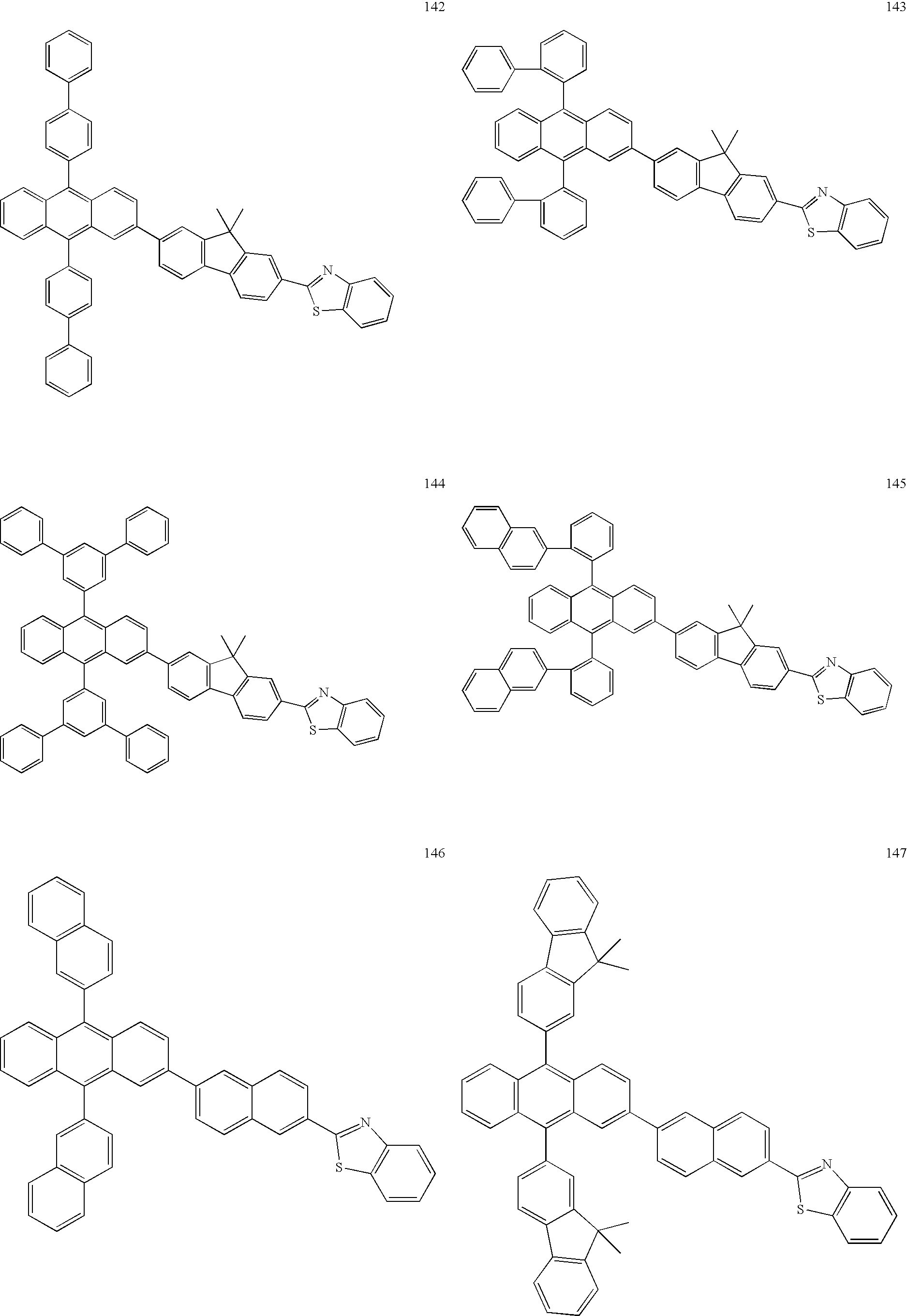

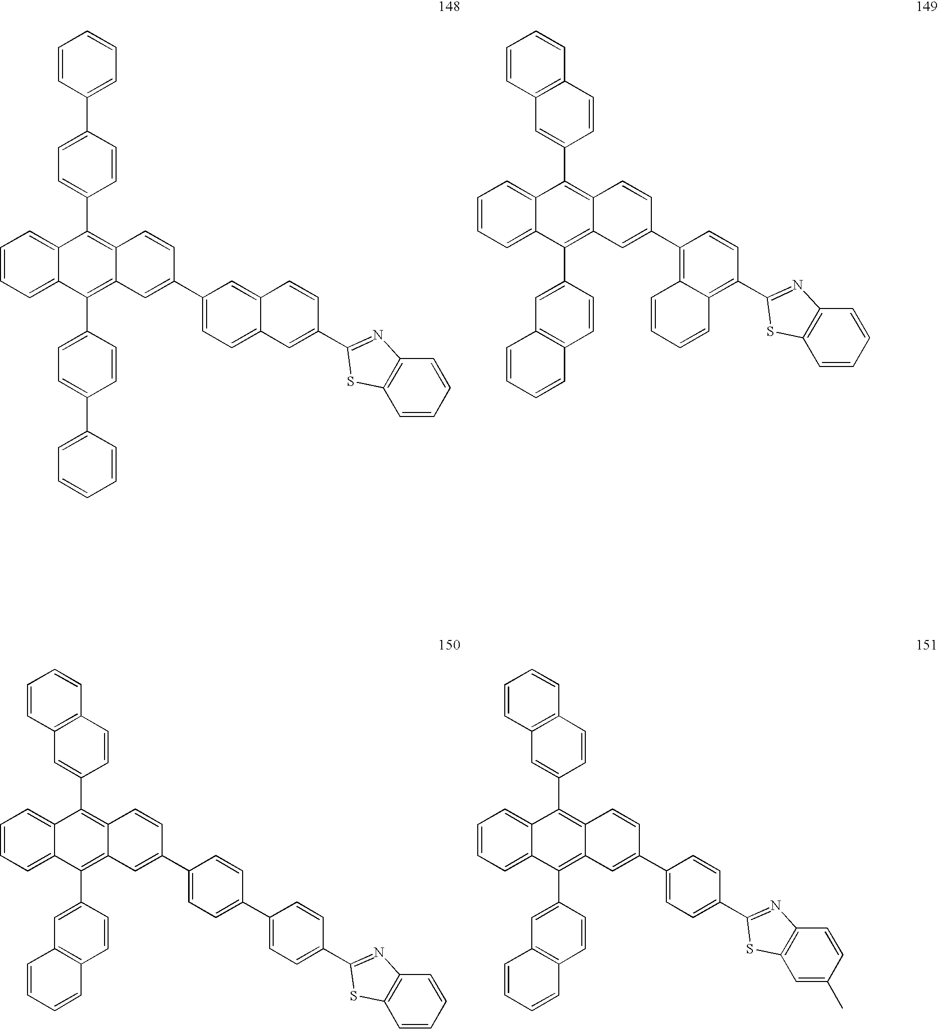

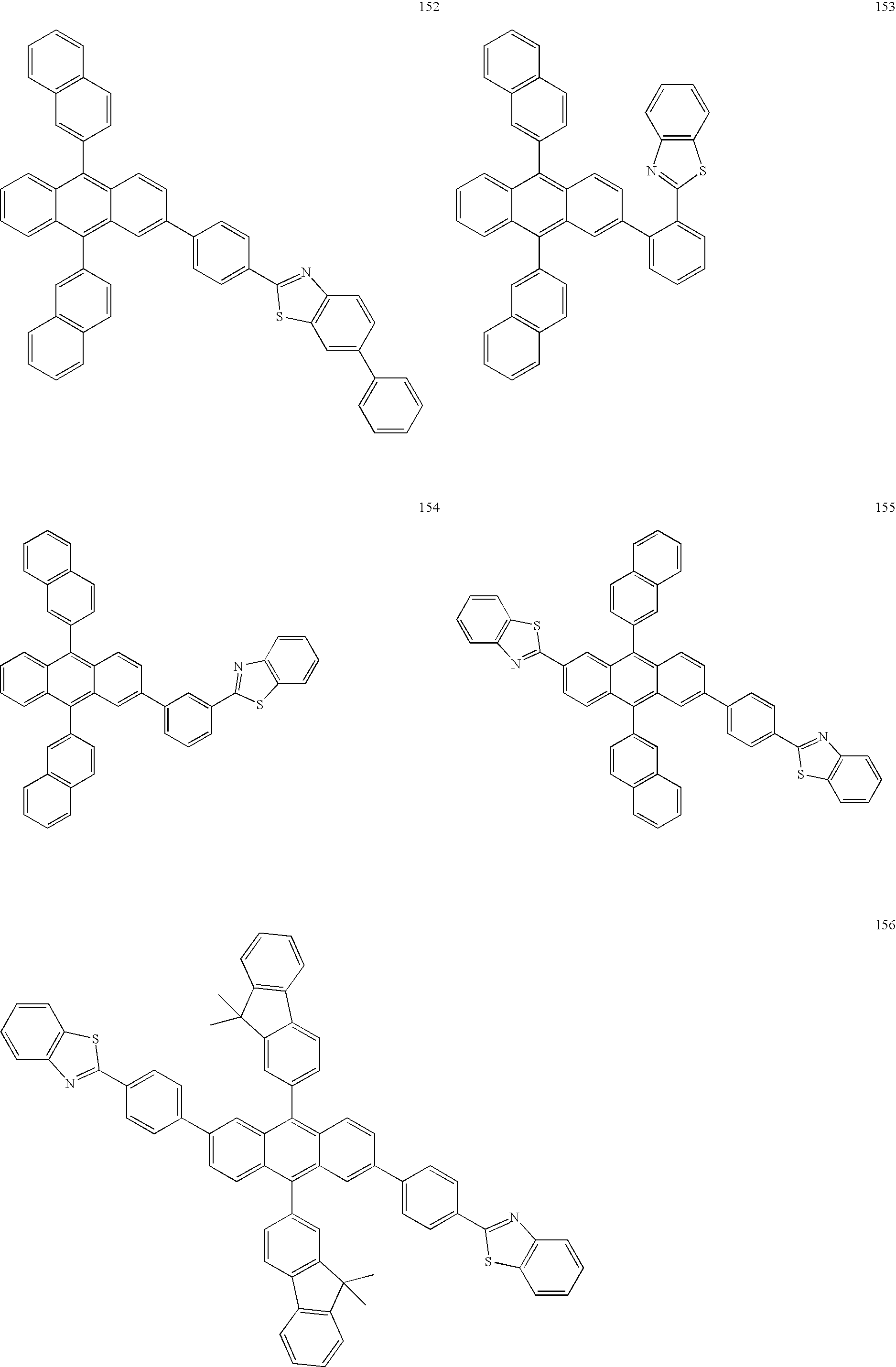

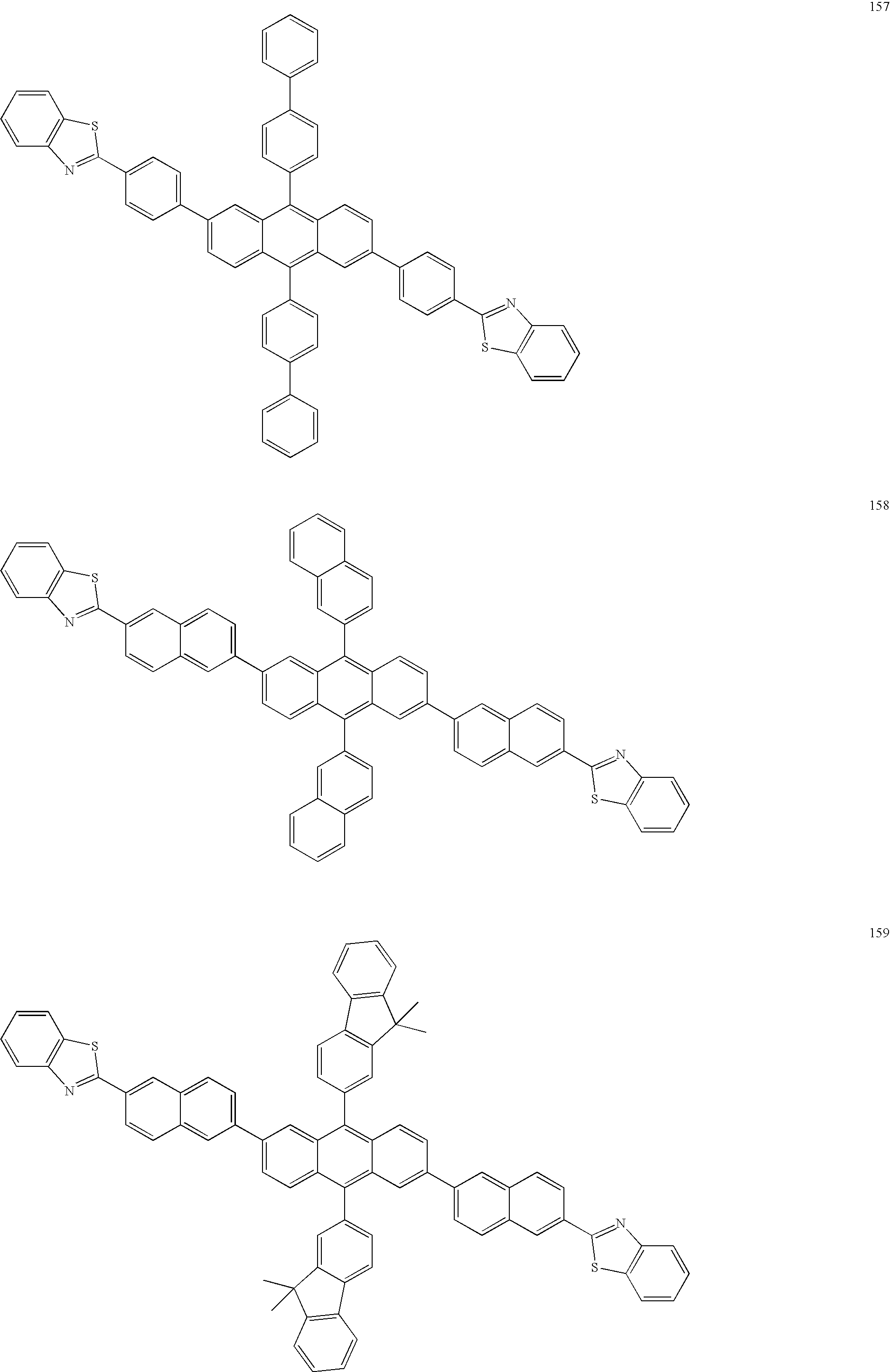

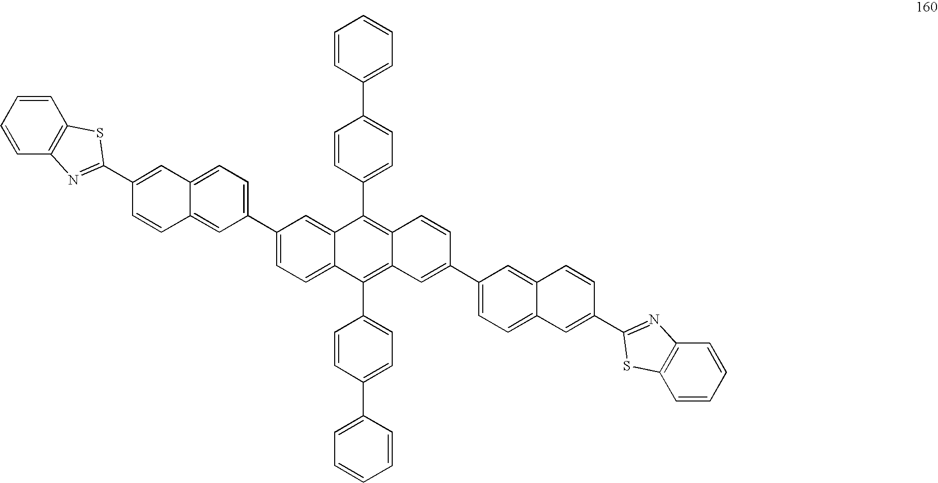

The thiazole system organic electroluminescent compounds according to the present invention can be prepared through the reaction route illustrated by Reaction Scheme (1):

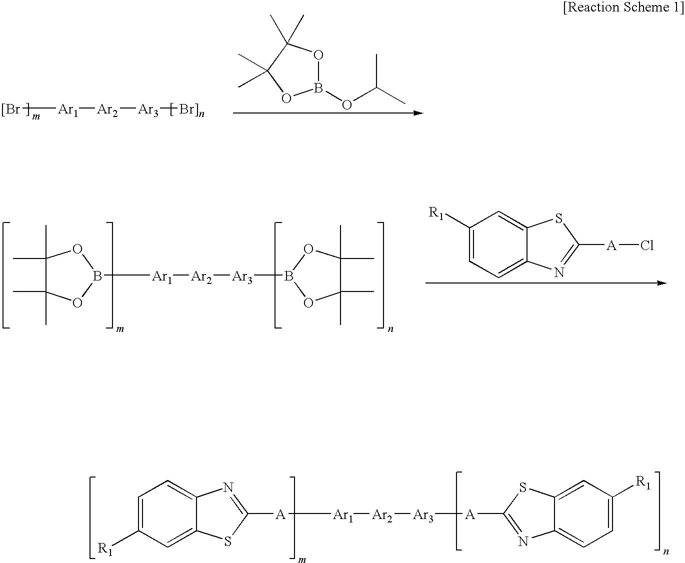

wherein, A, Ar₁, Ar₂, Ar₃, R₁, R₁₃, R₁₄, R₁₅, R₁₆, R₁₇, R₁₈, m and n are defined as for Chemical Formula (1) above.

Among the bromo compounds employed in Reaction Scheme (1) as the starting material, a reaction route for 9,10-bis(2-bromophenyl)anthracene, for instance, is illustrated by Reaction Scheme (2), but it is not restricted thereto.

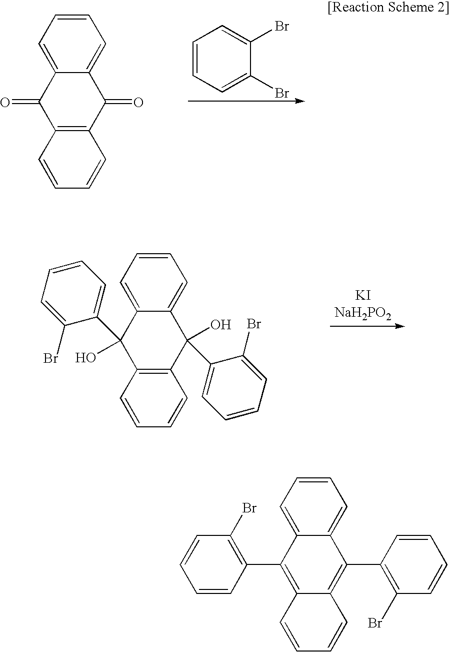

The starting material, dione or mono-one compound, for preparing the bromo compound in Reaction Scheme (2) may have further halogen atom(s) such as bromine. The reaction route for preparing a thiazole system organic electroluminescent compound according to the present invention starting from the dione or mono-one compound having halogen substituent(s) can be illustrated by Reaction Scheme (3), but it is not restricted thereto.

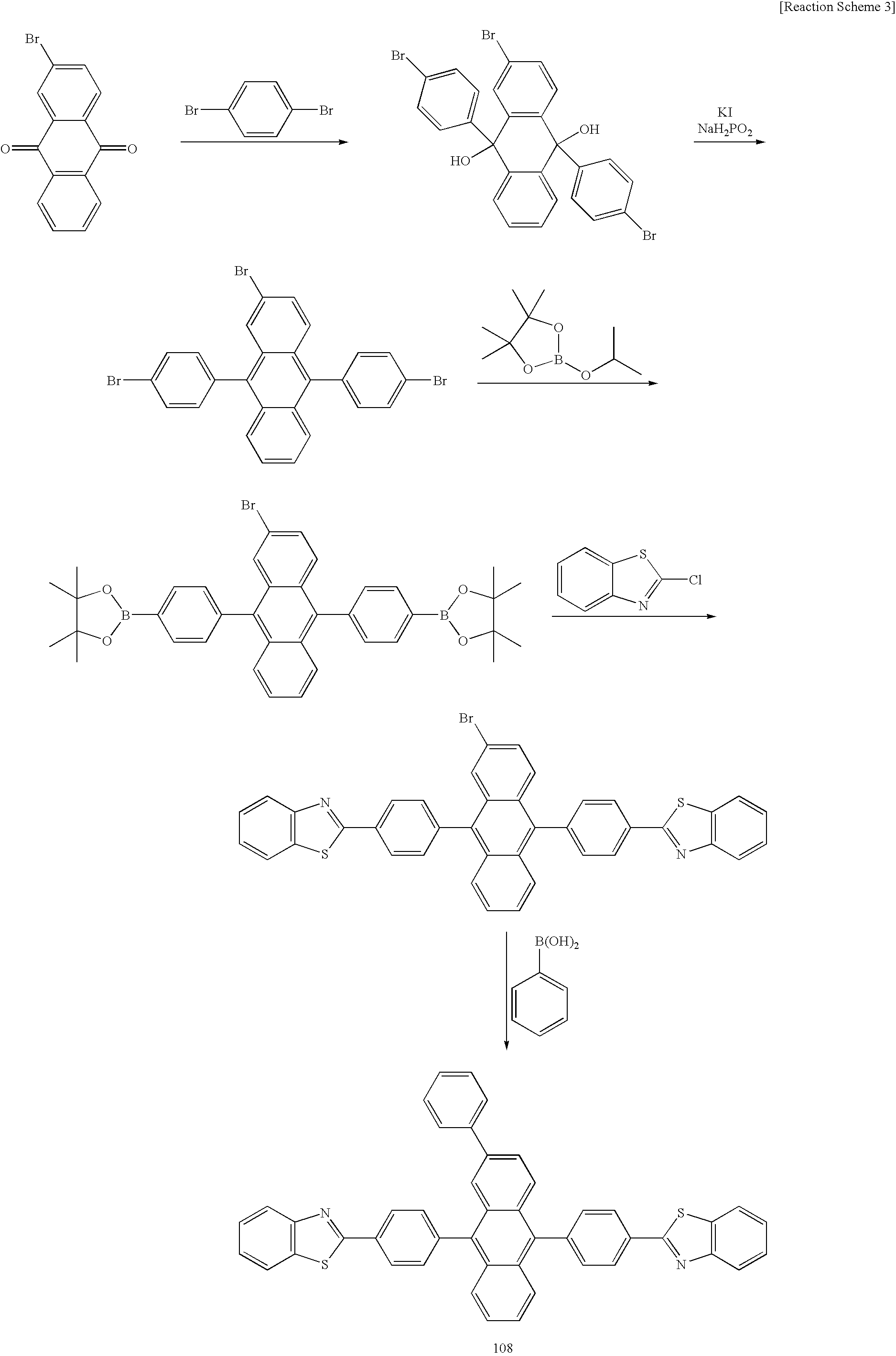

### DESCRIPTION OF DRAWINGS

FIG. 1 is a cross-sectional view of an OLED;

FIG. 2 shows luminous efficiency curve of Alq:C545T as a conventional electroluminescent material;

FIG. 3 shows luminous efficiency curve of Example 10 (Compound 109);

FIG. 4 shows luminance-voltage curve comparing Example 10 (Compound 109) and Comparative Example 1; and

FIG. 5 shows power efficiency-luminance curve comparing Example 10 (Compound 109) and Comparative Example 1.

### DESCRIPTION OF SYMBOLS OF SIGNIFICANT PARTS OF THE DRAWINGS

1: Glass
2: Transparent electrode
3: Hole injection layer
4: Hole transportation layer
5: Electroluminescent layer
6: Electron transportation layer
7: Electron injection layer
8: Al cathode

### BEST MODE

The present invention is further described with respect to the novel thiazole system organic electroluminescent compounds according to the present invention, processes for preparing the same and the electroluminescent properties of the device employing the same, by referring to Preparation Examples and Examples, which are provided for illustration only but are not intended to be limiting in any way.

### Preparation Examples

### Preparation Example 1

### Preparation of Compound (100)

Under nitrogen atmosphere, 1,2-dibromobenzene (56.7 g, 240.1 mmol) was dissolved in tetrahydrofuran (500 mL), and n-butyllithium (2.5M solution in n-hexane) (115.3 mL, 288.2 mmol) was slowly added thereto at −78° C. After stirring the mixture for 2 hours, anthracene-9,10-dione (20.0 g, 96.1 mmol) was added thereto, and the resultant mixture was slowly warmed to room temperature, and stirred for 16 hours. Then the reaction mixture was extracted with water and ethyl acetate. The extract was dried under reduced pressure, and recrystallized from ethyl acetate (300 mL) and n-hexane (500 mL) to obtain 9,10-bis(2-bromophenyl)-9,10-dihydroanthracene-9,10-diol (35.1 g, 67.2 mmol).

The compound, 9,10-bis(2-bromophenyl)-9,10-dihydroanthracene-9,10-diol (35.1 g, 67.2 mmol) thus obtained, potassium iodide (44.7 g, 268.9 mmol) and sodium hydrophosphite (57.0 g, 537.9 mmol) were dissolved in acetic acid (500 mL), and the solution was stirred at 100° C. under reflux for 18 hours. After cooling the reaction mixture to room temperature, water (150 mL) was slowly added thereto to quench the reaction. The reaction was extracted with dichloromethane, and the extract was dried under reduced pressure. Recrystallization from methanol (300 mL) and ethyl acetate (100 mL) gave 9,10-bis(2-bromophenyl)anthracene (29.5 g, 60.5 mmol).

Under nitrogen atmosphere, 9,10-bis(2-bromophenyl)anthracene (29.5 g, 60.5 mmol) was dissolved in tetrahydrofuran (400 mL), and n-buthyllithium (2.5M solution in n-hexane) (16.8 mL, 181.7 mmol) was slowly added thereto at −78° C., and the mixture was stirred for 2 hours. While maintaining the temperature, 2-isopropoxy-4,4,5,5-tetramethyl-1,3,2-oxaborolane (49.4 mL, 24.2 mmol) was added thereto. After warming to room temperature, the reaction mixture was stirred for 2 hours. The reaction mixture was extracted with ethyl acetate (300 mL) and dried under reduced pressure. Recrystallization from ethyl acetate (300 mL) and n-hexane (500 mL) gave 4,4,5,5-tetramethyl-2-(2-(10-(2 (4,4,5,5-tetramethyl-1,3,2-dioxaborolan-2-yl)phenyl)anthracen-9-yl)phenyl)-1,3,2-dioxaborolane (17.6 g, 30.3 mmol).

The compound, 4,4,5,5-tetramethyl-2-(2-(10-(2-(4,4,5,5-tetramethyl-1,3,2-dioxaborolan-2-yl)phenyl)anthracen-9-yl)phenyl)-1,3,2-dioxaborolane (17.6 g, 30.3 mmol), 2-chlorobenzo[d]thiazole (15.2 mL, 121.0 mmol), Aliquat 336 (6.9 mL, 15.1 mmol), tetrakis(triphenylphosphine)palladium (0) (Pd(PPh₃)₄) (17.5 g, 15.1 mmol), and 2M potassium carbonate (16.7 g, 121.0 mmol) were dissolved in toluene (300 mL), and the solution was stirred under reflux at 120° C. for 6 hours. After cooling the reaction mixture to room temperature, water was slowly added thereto to quench the reaction. The solid produced was filtered and washed with acetone to obtain the objective compound (100) (13.4 g, 22.5 mmol, overall yield: 23.4%).

¹H NMR (200 MHz, CDCl₃): δ 7.28-7.32 (m, 8H), 7.54-7.55 (m, 8H), 7.67 (d, 4H), 8.12 (d, 2H), 8.23 (s, 2H)

MS/FAB: 596.14 (found), 596.76 (calculated)

### Preparation Example 2

### Preparation of Compound (101)

According to the same procedure as in Preparation Example 1 but using anthracene-9,10-dione (20.0 g, 96.1 mmol), 1,3-dibromobenzene (56.7 g, 240.1 mmol) and 2-chlorobenzo[d]thiazole (16.7 mL, 133.3 mmol), obtained was Compound (101) (15.5 g, 26.0 mmol, overall yield: 27.1%).

¹H NMR (200 MHz, CDCl₃): δ 7.32-7.44 (m, 10H), 7.55 (t, 4H), 7.67-7.70 (m, 10H), 8.12 (d, 2H), 8.23 (d, 2H)

MS/FAB: 596.14 (found), 596.76 (calculated)

### Preparation Example 3

### Preparation of Compound (102)

According to the same procedure as in Preparation Example 1 but using anthracene-9,10-dione (20.0 g, 96.1 mmol), 1,3,5-tribromobenzene (75.6 g, 240.2 mmol) and 2-chlorobenzo[d]thiazole (23.7 mL, 188.9 mmol), obtained was the objective compound (102) (17.3 g, 20.1 mmol, overall yield: 20.9%).

¹H NMR (200 MHz, CDCl₃): δ 7.32 (m, 4H), 7.55 (t, 8H), 7.66-7.67 (m, 10H), 8.12 (d, 2H), 8.23 (d, 2H)

MS/FAB: 862.14 (found), 863.1 (calculated)

### Preparation Example 4

### Preparation of Compound (103)

According to the same procedure as in Preparation Example 1 but using anthracene-9,10-dione (20.0 g, 96.1 mmol), 1,4-dibromonaphthalene (68.7 g, 240.1 mmol) and 2-chlorobenzo[d]thiazole (13.0 mL, 103.7 mmol), obtained was the objective compound (103) (13.6 g, 19.5 mmol, overall yield: 20.0%).

¹H NMR (200 MHz, CDCl₃): δ 7.32 (m, 8H), 7.55 (t, 4H), 7.60 (s, 4H) 7.67 (m, 8H), 8.12 (d, 2H), 8.23 (d, 2H)

MS/FAB: 696.17 (found), 696.88 (calculated)

### Preparation Example 5

### Preparation of Compound (104)

According to the same procedure as in Preparation Example 1 but using anthracene-9,10-dione (20.0 g, 96.1 mmol), 2,6-dibromonaphthalene (68.7 g, 240.1 mmol) and 2-chlorobenzo[d]thiazole (13.3 mL, 105.5 mmol), obtained was the objective compound (104) (14.2 g, 20.3 mmol, overall yield: 21.1%).

¹H NMR (200 MHz, CDCl₃): δ 7.32 (m, 4H), 7.54-7.55 (m, 8H), 7.67 (m, 4H), 7.73 (d, 4H), 7.89 (s, 4H), 8.12 (d, 2H), 8.23 (d, 2H)

MS/FAB: 696.17 (found), 696.88 (calculated)

### Preparation Example 6

### Preparation of Compound (105)

In toluene (100 mL), dissolved were 2-chlorobenzo[d]thiazole (10.0 g, 59.0 mmol), 4-chlorophenylboronic acid (11.1 g, 70.7 mmol), and trans-dichlorobis(triphenylphosphine)palladium (II) (Pd(PPh₃)₂Cl₂ (4.2 g, 5.9 mmol). To the solution, added was 2M sodium carbonate solution (87 mL), and the resultant mixture was stirred under reflux for 3 hours.

After the reaction was completed, the reaction mixture was cooled to room temperature, and extracted with ethyl acetate (300 mL). The extract was dried under reduced pressure and recrystallized from dichloromethane (100 mL) and hexane (100 mL) to obtain 2-(4-chlorophenyl)benzo[d]thiazole (13.0 g, 53.1 mmol, yield: 90%).

According to the same procedure as in Preparation Example 1 but using anthracene-9,10-dione (20.0 g, 96.1 mmol), 1,2-dibromobenzene (56.7 g, 240.1 mmol) and 2-(4-chlorophenyl)benzo[d]thiazole (29.7 g, 120.9 mmol), obtained was the objective compound (105) (13.1 g, 17.5 mmol, overall yield: 18.2%).

¹H NMR (200 MHz, CDCl₃): δ 7.28-7.32 (m, 8H), 7.54-7.55 (m, 16H), 7.67 (m, 4H), 8.12 (d, 2H), 8.23 (d, 2H)

MS/FAB: 748.20 (found), 748.95 (calculated)

### Preparation Example 7

### Preparation of Compound (106)

According to the same procedure as in Preparation Example 1 but using anthracene-9,10-dione (20.0 g, 96.1 mmol), 1,4-dibromobenzene (56.7 g, 240.1 mmol) and 2-(4-chlorophenyl)benzo[d]thiazole (25.3 g, 103.0 mmol), obtained was the objective compound (106) (14.5 g, 19.4 mmol, overall yield: 20.2%).

¹H NMR (200 MHz, CDCl₃): δ 7.32 (m, 4H), 7.54-7.55 (m, 20H), 7.67 (m, 4H), 8.12 (d, 2H), 8.23 (d, 2H)

MS/FAB: 748.20 (found), 748.95 (calculated)

### Preparation Example 8

### Preparation of Compound (107)

According to the same procedure as in Preparation Example 1 but using anthracene-9,10-dione (20.0 g, 96.1 mmol), 1,3-dibromobenzene (56.7 g, 240.1 mmol) and 2-(4-chlorophenyl)benzo[d]thiazole (32.7 g, 133.3 mmol), obtained was the objective compound (107) (13.8 g, 18.4 mmol, overall yield: 19.2%).

¹H NMR (200 MHz, CDCl₃): δ 7.32-7.44 (m, 10H), 7.54-7.55 (m, 12H), 7.67-7.70 (m, 6H), 8.12 (d, 2H), 8.23 (d, 2H)

MS/FAB: 748.20 (found), 748.95 (calculated)

### Preparation Example 9

### Preparation of Compound (108)

According to the same procedure as in Preparation Example 1 but using 2-bromoanthracene-9,10-dione (27.6 g, 96.1 mmol), 1,4-dibromobenzene (56.7 g, 240.1 mmol) and 2-chlorobenzo[d]thiazole (19.8 ml, 103.4 mmol), obtained was 2-(4-(9-(4-(benzo[d]thiazol-2-yl)phenyl)-2-bromoanthracen-10-yl)phenyl)benzo[d]thiazole (15.5 g, 22.9 mmol).

In a mixture of toluene (300 mL) and ethanol (150 mL), dissolved were 2-(4-(9-(4-(benzo[d]thiazol-2-yl)phenyl)-2-bromoanthracen-10-yl)phenyl)benzo[d]thiazole (15.5 g, 22.9 mmol), phenylboronic acid (3.4 g, 27.5 mmol) and tetrakis(triphenylphosphine)palladium (0) (Pd(PPh₃)₄) (2.6 g, 2.3 mmol). Aqueous solution of sodium carbonate (2M Na₂CO₃) (250 mL) was added thereto, and the resultant mixture was stirred under reflux for 6 hours. After cooling to room temperature, water was slowly added to the reaction mixture to quench the reaction. The reaction mixture was extracted with dichloromethane (300 mL), and the extract was dried under reduced pressure, and recrystallized from ethyl acetate (200 mL) and methanol (100 mL) to obtain the objective compound (108) (13.9 g, 20.6 mmol, overall yield: 21.4%).

¹H NMR (200 MHz, CDCl₃): δ 7.22-7.32 (m, 5H), 7.48-7.55 (m, 15H), 7.67-7.73 (m, 3H), 7.89 (s, 1H) 8.12 (d, 2H), 8.23 (d, 2H)

MS/FAB: 672.17 (found), 672.86 (calculated)

### Preparation Example 10

### Preparation of Compound (109)

According to the same procedure as in Preparation Example 9 but using 2-(4-(9-(4-(benzo[d]thiazol-2-yl)phenyl)-2-bromo-anthracen-10-yl)phenyl)benzo[d]thiazole (15.5 g, 22.9 mmol), 2-naphthaleneboronic acid (5.9 g, 34.4 mmol), tetrakis(triphenylphosphine)palladium (0) (Pd (PPh₃)₄) (2.6 g, 2.3 mmol), 2M sodium carbonate (Na₂CO₃) (12.1 g, 114.5 mmol) and toluene (300 mL), obtained was the objective compound (109) (13.2 g, 18.3 mmol, overall yield: 19.0%).

¹H NMR (200 MHz, CDCl₃): δ 7.32 (m, 4H), 7.54-7.55 (m, 14H), 7.67 (m, 4H), 7.73 (d, 2H), 7.89 (s, 2H), 8.12 (d, 2H), 8.23 (d, 2H)

MS/FAB: 722.19 (found), 722.92 (calculated)

### Preparation Example 11

### Preparation of Compound (110)

In tetrahydrofuran (300 mL), dissolved was 2-(4-(9-(4-(benzo[d]thiazol-2-yl)phenyl)-2-bromoanthracen-10-yl)phenyl)benzo[d]thiazole (15.5 g, 22.9 mmol) under nitrogen atmosphere, and the solution was chilled to −78° C. After slowly adding n-butyllithium (2.5M solution in n-hexane) (6.4 mL, 68.9 mmol) thereto, the mixture was stirred for one hour. While maintaining the temperature, chlorotrimethyl silane (7.5 g, 68.8 mmol) was added thereto. The reaction mixture was slowly warmed to room temperature, and stirred for 24 hours. Then sodium chloride solution (50 mL) was added thereto to quench the reaction, and the resultant mixture was extracted with ethyl acetate (300 mL). The extract was dried under reduced pressure and recrystallized from ethyl acetate (200 mL) and methanol (100 mL) to obtain the objective compound (110) (6.9 g, 10.3 mmol, overall yield: 10.7%).

¹H NMR (200 MHz, CDCl₃): δ 0.66 (s, 9H), 7.32 (m, 2H), 7.54-7.55 (m, 13H), 7.65-7.67 (m, 3H), 7.89 (s, 1H), 8.12 (d, 2H), 8.23 (d, 2H)

MS/FAB: 668.12 (found), 668.94 (calculated)

### Preparation Example 12

### Preparation of Compound (111)

According to the same procedure as in Preparation Example 11 but using 2-(4-(9-(4-(benzo[d]thiazol-2-yl)phenyl)-2-bromo-anthracen-10-yl)phenyl)benzo[d]thiazole (15.5 g, 22.9 mmol), chlorotriphenylsilane (10.1 g, 34.3 mmol), n-butyllithium (2.5M solution in n-hexane) (6.4 mL, 68.9 mmol) and tetrahydrofuran (300 mL), obtained was the objective compound (III) (7.8 g, 9.1 mmol, overall yield: 9.5%).

¹H NMR (200 MHz, CDCl₃): δ 7.32-7.36 (m, 11H), 7.54-7.55 (m, 18H), 7.60-7.67 (m, 3H), 7.77 (d, 1H), 7.94 (s, 1H), 8.12 (d, 2H), 8.23 (d, 2H)

MS/FAB: 854.22 (found), 855.15 (calculated)

### Preparation Example 13

### Preparation of Compound (112)

According to the same procedure as in Preparation Example 1 but using 2-methylanthraquinone (21.4 g, 96.1 mmol), 1,4-dibromobenzene (56.7 g, 240.1 mmol) and 2-chlorobenzo[d]thiazole (19.8 mL, 103.4 mmol), obtained was the objective compound (112) (14.0 g, 22.9 mmol, overall yield: 23.8%).

¹H NMR (200 MHz, CDCl₃): δ 2.46 (s, 3H), 7.18 (d, 1H), 7.32 (m, 2H), 7.46 (s, 1H), 7.54-7.67 (m, 15H), 8.12 (d, 2H), 8.23 (d, 2H)

MS/FAB: 610.15 (found), 610.79 (calculated)

### Preparation Example 14

### Preparation of Compound (113)

According to the same procedure as in Preparation Example 1 but using 2,3-dimethylanthracene-9,10-dione (22.7 g, 96.1 mmol), 1,4-dibromobenzene (56.7 g, 240.1 mmol) and 2-chlorobenzo[d]thiazole (19.8 mL, 103.4 mmol), obtained was the objective compound (113) (13.1 g, 21.0 mmol, overall yield: 21.9%).

¹H NMR (200 MHz, CDCl₃): δ 2.46 (s, 6H), 7.32 (m, 2H), 7.40 (s, 2H), 7.54-7.55 (m, 12H), 7.67 (m, 2H), 8.12 (d, 2H), 8.23 (d, 2H)

MS/FAB: 624.17 (found), 624.82 (calculated)

### Preparation Example 15

### Preparation of Compound (114)

According to the same procedure as in Preparation Example 1 but using 2-tert-butylanthracene-9,10-dione (25.4 g, 96.1 mmol), 1,4-dibromobenzene (56.7 g, 240.1 mmol) and 2-chlorobenzo[d]thiazole (19.8 mL, 103.4 mmol), obtained was the objective compound (114) (12.2 g, 18.7 mmol, overall yield: 19.5%).

¹H NMR (200 MHz, CDCl₃): δ 1.40 (s, 9H), 7.18 (d, 1H), 7.32 (m, 2H), 7.46 (s, 1H), 7.54-7.55 (m, 12H), 7.61-7.67 (m, 3H), 8.12 (d, 2H), 8.23 (d, 2H)

MS/FAB: 652.2 (found), 652.87 (calculated)

### Preparation Example 16

### Preparation of Compound (115)

According to the same procedure as in Preparation Example 1 but using anthracene-9,10-dione (20.0 g, 96.1 mmol), 1,4-dibromobenzene (56.7 g, 240.1 mmol) and 2-chloro-6-(trimethylsilyl)benzo[d]thiazole (24.9 g, 103.0 mmol), obtained was the objective compound (115) (3.8 g, 5.2 mmol, overall yield: 5.4%).

¹H NMR (200 MHz, CDCl₃): δ 0.66 (s, 9H), 7.32 (m, 4H), 7.54 (s, 8H), 7.67 (m, 4H), 7.77 (d, 2H), 8.12 (d, 2H), 8.23 (d, 2H)

MS/FAB: 740.22 (found), 741.12 (calculated)

### Preparation Example 17

### Preparation of Compound (116)

According to the same procedure as in Preparation Example 1 but using anthracene-9,10-dione (20.0 g, 96.1 mmol), 1,4-dibromobenzene (56.7 g, 240.1 mmol) and 2-chloro-6-(trifluoromethyl)benzo[d]thiazole (24.5 g, 103.0 mmol), obtained was the objective compound (116) (8.2 g, 11.2 mmol, overall yield: 11.6%).

¹H NMR (200 MHz, CDCl₃): δ 7.32 (m, 4H), 7.54 (s, 8H), 7.67 (m, 4H), 7.74 (d, 2H), 8.16 (d, 2H), 8.31 (s, 2H)

MS/FAB: 732.11 (found), 732.76 (calculated)

### Preparation Example 18

### Preparation of Compound (117)

According to the same procedure as in Preparation Example 1 but using anthracene-9,10-dione (20.0 g, 96.1 mmol), 1,4-dibromobenzene (56.7 g, 240.1 mmol) and 2-chloro-6-phenylbenzo[d]thiazole (25.3 g, 103.0 mmol), obtained was the objective compound (117) (15.1 g, 20.1 mmol, overall yield: 20.9%).

¹H NMR (200 MHz, CDCl₃): δ 7.22-7.32 (m, 10H), 7.48 (d, 4H), 7.54 (s, 8H), 7.67 (m, 4H), 7.77 (s, 2H), 8.29 (d, 2H), 8.34 (s, 2H)

MS/FAB: 748.20 (found), 748.95 (calculated)

### Preparation Example 19

### Preparation of Compound (118)

According to the same procedure as in Preparation Example 1 but using anthracene-9,10-dione (20.0 g, 96.1 mmol), 2,7-dibromo-9,9-dimethyl-9H-fluorene (84.5 g, 240.1 mmol) and 2-chlorobenzo[d]thiazole (8.2 mL, 65.2 mmol), obtained was the objective compound (118) (9.5 g, 11.4 mmol, overall yield: 11.9%).

¹H NMR (200 MHz, CDCl₃): δ 1.67 (s, 12H), 7.32 (m, 4H), 7.55-7.67 (m, 12H), 7.77 (s, 4H), 7.90 (d, 4H), 8.12 (t, 2H), 8.23 (t, 2H)

MS/FAB: 828.26 (found), 829.08 (calculated)

### Preparation Example 20

### Preparation of Compound (119)

According to the same procedure as in Preparation Example 1 but using 9-(10-oxoanthracen-9(10H)-ylidene)anthracen-10(9H)-one (36.9 g, 96.1 mmol), 1,4-dibromobenzene (56.7 g, 240.3 mmol) and 2-chlorobenzo[d]thiazole (13.0 mL, 103.2 mmol), obtained was the objective compound (119) (15.0 g, 19.4 mmol, overall yield: 20.2%).

¹H NMR (200 MHz, CDCl₃): δ 7.32 (m, 12H), 7.54-7.55 (m, 12H), 7.67 (t, 8H), 8.12 (d, 2H), 8.23 (d, 2H)

MS/FAB: 772.2 (found), 772.98 (calculated)

### Preparation Example 21

### Preparation of Compound (120)

According to the same procedure as in Preparation Example 1 but using 9-(10-oxoanthracen-9(10H)-ylidene)anthracen-10(9H)-one (36.9 g, 96.1 mmol), 1,3-dibromobenzene (56.7 g, 240.1 mmol) and 2-chlorobenzo[d]thiazole (16.7 mL, 133.3 mmol), obtained was the objective compound (120) (20.1 g, 26.0 mmol, overall yield: 27.1%).

¹H NMR (200 MHz, CDCl₃): δ 7.32-7.44 (m, 14H), 7.55 (t, 4H), 7.67-7.70 (m, 10H), 8.12 (d, 2H), 8.23 (d, 2H)

MS/FAB: 772.2 (found), 772.98 (calculated)

### Preparation Example 22

### Preparation of Compound (121)

According to the same procedure as in Preparation Example 1 but using 9-(10-oxoanthracen-9(10H)-ylidene)anthracen-10(9H)-one (36.9 g, 96.1 mmol), 1,3,5-tribromobenzene (75.6 g, 240.2 mmol) and 2-chlorobenzo[d]thiazole (23.7 mL, 188.9 mmol), obtained was the objective compound (121) (20.9 g, 20.1 mmol, overall yield: 20.9%).

¹H NMR (200 MHz, CDCl₃): δ 7.32 (m, 8H), 7.55 (m, 8H), 7.66-7.67 (m, 14H), 8.12 (d, 4H), 8.23 (d, 4H)

MS/FAB: 1038.2 (found), 1039.32 (calculated)

### Preparation Example 23

### Preparation of Compound (122)

According to the same procedure as in Preparation Example 1 but using 9-(10-oxoanthracen-9(10H)-ylidene)anthracen-10(9H)-one (36.9 g, 96.1 mmol), 1,2-dibromobenzene (56.7 g, 240.1 mmol) and 2-(4-chlorophenyl)benzo[d]thiazole (13.3 mL, 105.5 mmol), obtained was the objective compound (122) (17.7 g, 20.3 mmol, overall yield: 21.1%).

¹H NMR (200 MHz, CDCl₃): δ 7.28-7.32 (m, 12H), 7.54-7.55 (m, 16H), 7.67 (m, 8H), 8.12 (d, 2H), 8.23 (d, 2H)

MS/FAB: 924.26 (found), 925.17 (calculated)

### Preparation Example 24

### Preparation of Compound (123)

According to the same procedure as in Preparation Example 1 but using 9-(10-oxoanthracen-9(10H)-ylidene)anthracen-10(9H)-one (36.9 g, 96.1 mmol), 2,6-dibromonaphthalene (68.7 g, 240.1 mmol) and 2-chlorobenzo[d]thiazole (13.3 mL, 105.5 mmol), obtained was the objective compound (123) (17.7 g, 20.3 mmol, overall yield: 21.1%).

¹H NMR (200 MHz, CDCl₃): δ 7.32 (m, 8H), 7.54-7.55 (m, 8H), 7.67-7.73 (m, 12H), 7.89 (s, 4H), 8.12 (d, 2H), 8.23 (d, 2H)

MS/FAB: 872.23 (found), 873.09 (calculated)

### Preparation Example 25

### Preparation of Compound (124)

According to the same procedure as in Preparation Example 1 but using 9-(10-oxoanthracen-9(10H)-ylidene)anthracen-10(9H)-one (36.9 g, 96.1 mmol), 1,4-dibromobenzene (56.7 g, 240.3 mmol) and 2-chloro-6-(trifluoromethyl)benzo[d]thiazole (24.5 g, 103.2 mmol), obtained was the objective compound (124) (17.6 g, 19.4 mmol, overall yield: 20.2%).

¹H NMR (200 MHz, CDCl₃): δ 7.32 (m, 8H), 7.54 (s, 8H), 7.67 (m, 8H), 7.74 (d, 2H), 8.16 (d, 2H), 8.31 (s, 2H)

MS/FAB: 908.18 (found), 908.97 (calculated)

### Preparation Example 26

### Preparation of Compound (125)

According to the same procedure as in Preparation Example 1 but using 9-(10-oxoanthracen-9(10H)-ylidene)anthracen-10(9H)-one (36.9 g, 96.1 mmol), 1,4-dibromobenzene (56.7 g, 240.3 mmol) and 2-chloro-6-(trimethylsilyl)benzo[d]thiazole (25.0 g, 103.2 mmol), obtained was the objective compound (125) (16.7 g, 18.2 mmol, overall yield: 18.9%).

¹H NMR (200 MHz, CDCl₃): δ 0.66 (s, 18H), 7.32 (m, 8H), 7.54 (s, 8H), 7.67 (m, 8H), 7.77 (d, 2H), 8.21 (d, 2H), 8.34 (s, 2H)

MS/FAB: 916.28 (found), 917.34 (calculated)

### Preparation Example 27

### Preparation of Compound (126)

According to the same procedure as in Preparation Example 1 but using 9-(10-oxoanthracen-9(10H)-ylidene)anthracen-10(9H)-one (36.9 g, 96.1 mmol), 1,4-dibromobenzene (56.7 g, 240.3 mmol) and 2-chloro-6-phenylbenzo[d]thiazole (25.3 g, 103.0 mmol), obtained was the objective compound (126) (10.4 g, 11.2 mmol, overall yield: 11.6%).

¹H NMR (200 MHz, CDCl₃): δ 7.32 (m, 14H), 7.48 (d, 4H), 7.54 (s, 8H), 7.67 (m, 8H), 7.77 (d, 2H), 8.29 (d, 2H), 8.34 (s, 2H)

MS/FAB: 924.26 (found), 925.17 (calculated)

### Preparation Example 28

### Preparation of Compound (127)

According to the same procedure as in Preparation Example 1 but using 9-(10-oxoanthracen-9(10H)-ylidene)anthracen-10(9H)-one (36.9 g, 96.1 mmol), 1,3-dibromobenzene (56.7 g, 240.1 mmol) and 2-chloro-6-(trimethylsilyl)benzo[d]thiazole (25.0 g, 103.4 mmol), obtained was the objective compound (127) (16.7 g, 18.2 mmol, overall yield: 18.9%).

¹H NMR (200 MHz, CDCl₃): δ 0.66 (s, 18H), 7.30-7.38 (m, 10H), 7.42-7.45 (m, 4H), 7.65-7.68 (m, 10H), 8.21-8.12 (m, 2H), 8.34 (s, 2H)

MS/FAB: 916.28 (found), 917.34 (calculated)

### Preparation Example 29

### Preparation of Compound (128)

According to the same procedure as in Preparation Example 1 but using anthracene-9,10-dione (20.0 g, 96.1 mmol), 1,3-dibromobenzene (56.7 g, 240.1 mmol) and 2-chloro-6-(trimethylsilyl)benzo[d]thiazole (32.2 g, 133.2 mmol), obtained was the objective compound (128) (13.8 g, 18.6 mmol, overall yield: 7.7%).

¹H NMR (200 MHz, CDCl₃): δ 0.66 (s, 18H), 7.32-7.38 (m, 6H), 7.42-7.46 (m, 4H), 7.67-7.70 (m, 6H), 7.77 (d, 2H), 8.21 (d, 2H), 8.34 (s, 2H)

MS/FAB: 740.22 (found), 741.12 (calculated)

### Preparation Example 30

### Preparation of Compound (129)

According to the same procedure as in Preparation Example 1 but using 2-methylanthraquinone (21.4 g, 96.1 mmol), 1,4-dibromobenzene (56.7 g, 240.1 mmol) and 2-chloro-6-(trimethylsilyl)benzo[d]thiazole (25.1 g, 103.8 mmol), obtained was the objective compound (129) (14.0 g, 19.8 mmol, overall yield: 23.8%).

¹H NMR (200 MHz, CDCl₃): δ 0.67 (s, 18H), 2.43 (s, 3H), 7.20-7.29 (m, 3H), 7.45 (s, 1H), 7.53-7.56 (m, 8H), 7.61-7.65 (m, 3H), 7.75-7.77 (d, 2H), 8.19-8.21 (d, 2H), 8.32 (d, 2H)

MS/FAB: 754.23 (found), 755.15 (calculated)

### Preparation Example 31

### Preparation of Compound (130)

According to the same procedure as in Preparation Example 1 but using 2-tert-butylanthracene-9,10-dione (25.4 g, 96.1 mmol), 1,4-dibromobenzene (56.7 g, 240.1 mmol) and 2-chloro-6-(trimethylsilyl)benzo[d]thiazole (25.0 g, 103.4 mmol), obtained was the objective compound (130) (12.2 g, 15.3 mmol, overall yield: 19.5%).

¹H NMR (200 MHz, CDCl₃): δ 0.65 (s, 18H), 1.42 (m, 3H), 7.16-7.28 (m, 3H), 7.45 (s, 1H), 7.50-7.54 (m, 8H), 7.62-7.66 (m, 3H), 7.74-7.77 (d, 2H), 8.19-8.21 (d, 2H), 8.34 (d, 2H)

MS/FAB: 796.28 (found), 797.23 (calculated)

### Preparation Example 32

### Preparation of Compound (131)

According to the same procedure as in Preparation Example 1 but using 2,3-dimethylanthracene-9,10-dione (22.7 g, 96.1 mmol), 1,4-dibromobenzene (56.7 g, 240.1 mmol) and 2-chloro-6-(trimethylsilyl)benzo[d]thiazole (24.4 g, 100.9 mmol), obtained was the objective compound (131) (13.1 g, 17.1 mmol, overall yield: 21.9%).

¹H NMR (200 MHz, CDCl₃): δ 0.67 (s, 18H), 2.47 (s, 6H), 7.31 (d, 2H), 7.41 (m, 2H), 7.56 (d, 8H), 7.66 (m, 2H), 8.21 (d, 2H), 8.34 (d, 2H)

MS/FAB: 768.25 (found), 769.18 (calculated)

### Preparation Example 33

### Preparation of Compound (132)

According to the same procedure as in Preparation Example 1 but using anthracene-9,10-dione (20.0 g, 96.1 mmol), 1,4-dibromobenzene (56.7 g, 240.1 mmol) and 2-chlorobenzo[d]thiazole (17.5 mL, 103.0 mmol), obtained was the objective compound (132) (8.2 g, 11.2 mmol, overall yield: 11.6%).

¹H NMR (200 MHz, CDCl₃): δ 7.32 (m, 4H), 7.54-7.55 (m, 12H), 7.67 (m, 4H), 8.12 (d, 2H), 8.23 (d, 2H)

MS/FAB: 597.74 (found), 596.14 (calculated)

### Preparation Example 34

### Preparation of Compound (133)

Under nitrogen atmosphere, 9-bromoanthracene (20.0 g, 77.8 mmol) was dissolved in tetrahydrofuran (200 mL), and the solution was chilled to −78° C. To the solution, n-butyllithium (n-BuLi, 2.5M in hexane) (37.4 mL, 93.4 mmol) was slowly added dropwise. After 30 minutes, trimethylborate (17.7 mL, 155.6 mmol) was added dropwise thereto. The temperature was slowly raised, and the mixture was further stirred at room temperature for one day. To the mixture, 1N aqueous hydrochloric acid (200 mL) was added, and the resultant mixture was stirred for minutes, and extracted with water (300 mL) and dichloromethane (200 mL). The extract was dried under reduced pressure and recrystallized from ethyl acetate (30 mL) and hexane (500 mL) to obtain 9-anthraceneboronic acid (9.3 g, 41.9 mmol).

To a mixture of toluene (200 mL) and ethanol (100 mL), dissolved were 9-anthraceneboronic acid (9.3 g, 41.9 mmol), 2-(4-chlorophenyl)benzo[d]thiazole (29.7 g, 120.9 mmol) and tetrakis(triphenylphosphine)palladium (0) (Pd(PPh₃)₄) (2.1 g, 1.8 mmol). To the solution, added was aqueous 2M sodium carbonate solution (100 mL), and the mixture was stirred at 120° C. under reflux for 12 hours. Then, the temperature was lowered to 25° C., and the reaction was quenched by adding distilled water (100 mL). The reaction mixture was extracted with ethyl acetate (100 mL), and the extract was dried under reduced pressure. Recrystallization from tetrahydrofuran (20 mL) and methanol (300 mL) gave 2-(4-anthracen-10-yl)phenyl)benzo[d]thiazole (6.5 g, 15.1 mmol).

Under nitrogen atmosphere, 2-(4-anthracen-10-yl)phenyl)benzo[d]thiazole (6.5 g, 15.1 mmol) and N-bromosuccinimide (3.0 g, 16.6 mmol) were dissolved in dichloromethane (200 mL), and the solution was stirred at 25° C. for one day. After quenching the reaction by adding distilled water (200 mL), the reaction mixture was extracted with dichloromethane (100 mL). The extract was dried under reduced pressure, and recrystallized from tetrahydrofuran (20 mL) and methanol (200 mL) to obtain 2-(4-(10-bromoanthracen-9-yl)phenyl)benzo[d]thiazole (6.9 g, 13.5 mmol).

In a mixture of toluene (150 mL) and ethanol (70 mL), dissolved were 2-(4-(10-bromoanthracen-9-yl)phenyl)benzo[d]thiazole (6.9 g, 13.5 mmol), 2-naphthylboronic acid (2.0 g, 16.2 mmol) and tetrakis(triphenylphosphine)palladium (O) (Pd(PPh₃)₄) (1.6 g, 1.4 mmol). Then, aqueous 2M sodium carbonate solution (70 mL) was added thereto, and the resultant mixture was stirred at 120° C. under reflux for 6 hours. After cooling the mixture to 25° C., the reaction was quenched by adding distilled water (100 mL). The reaction mixture was extracted with ethyl acetate (100 mL), and the extract was dried under reduced pressure. Recrystallization from tetrahydrofuran (20 mL) and methanol (200 mL) gave the objective compound (133) (5.8 g, 11.4 mmol).

¹H NMR (200 MHz, CDCl₃): δ 7.32 (m, 6H), 7.54-7.55 (m, 7H), 7.67-7.73 (m, 7H), 7.89 (d, 2H), 8.12-8.23 (m, 2H)

MS/FAB: 514.16 (found), 513.16 (calculated)

### Preparation Example 35

### Preparation of Compound (134)

Under nitrogen atmosphere, 2-(4-chlorophenyl)benzo[d]thiazole (20.0 g, 68.9 mmol) was dissolved in tetrahydrofuran (THF) (700 mL), and the solution was chilled to −78° C. To the solution, n-buthyllithium (2.5M solution in n-hexane) (33.0 mL, 82.7 mmol) was slowly added, and the mixture was stirred for one hour. While maintaining the temperature at −78° C., trimethylborate (10.7 g, 103.3 mmol) was added thereto. Then the temperature was slowly raised, and the mixture was stirred at room temperature for 18 hours. Water (700 mL) was added thereto, and the resultant mixture was stirred for one hour, and extracted with ethyl acetate. The extract was dried under reduced pressure, and recrystallized from n-hexane (300 mL) to obtain 4-(benzo[d]thiazol-2-yl)phenylboronic acid (17.0 g, 66.6 mmol).

A solution of 2-chloroanthraquinone (6.8 g, 27.8 mmol), 4-(benzo[d]thiazol-2-yl)phenylboronic acid (10.6 g, 41.7 mmol) and tetrakis(triphenylphosphine)palladium (0) (Pd(PPh₃)₄) (3.2 g, 2.8 mmol) dissolved in 2M potassium carbonate (K₂CO₃) (150 mL), ethylene glycol dimethyl ether (DME) (300 mL) and ethanol (150 mL) was stirred under reflux for 20 hours. After cooling to room temperature, water (200 mL) was added to the reaction mixture, and the resultant mixture was stirred and extracted with ethyl acetate (300 mL). The extract was dried under reduced pressure, and recrystallized from n-hexane (300 mL) to obtain 2-(4-(benzo[d]thiazol-2-yl)phenyl)anthracene-9,10-dione (11.6 g, 27.7 mmol).

According to the same procedure as in Preparation Example 1 but using 2-bromonaphthalene (19.8 g, 95.8 mmol) and 2-(4-(benzo[d]thiazol-2-yl)phenyl)anthracene-9,10-dione (10.0 g, 24.0 mmol) under nitrogen atmosphere, obtained was the objective compound (134) (5.5 g, 8.5 mmol, overall yield: 42.5%).

¹H NMR (200 MHz, CDCl₃): δ 7.34-7.36 (m, 6H), 7.55-7.58 (m, 9H), 7.67-7.73 (m, 9H), 7.86-7.89 (m, 3H), 8.14 (d, 1H), 8.26 (d, 1H)

MS/FAB: 640.21 (found), 639.80 (calculated)

### Preparation Example 36

### Preparation of Compound (135)

According to the same procedure as in Preparation Example 1 but using 2-bromo-9,9-dimethylfluorene (13.7 g, 50.3 mmol) and 2-(4-(benzo[d]thiazol-2-yl)phenyl)anthracene-9,10-dione (7.0 g, 16.7 mmol), obtained was the objective compound (135) (5.0 g, overall yield: 30.2%).

¹H NMR (200 MHz, CDCl₃): δ 1.63 (d, 12H), 7.30-7.38 (m, 6H), 7.53-7.55 (m, 5H), 7.55-7.57 (m, 4H), 7.58-7.60 (m, 2H), 7.62-7.65 (m, 2H), 7.70-7.74 (m, 3H), 7.84-7.89 (m, 5H), 8.13 (d, 1H), 8.24 (d, 1H)

MS/FAB: 772.30 (found), 772.01 (calculated)

### Preparation Example 37

### Preparation of Compound (136)

According to the same procedure as in Preparation Example 1 but using 4-bromobiphenyl (11.7 g, 50.3 mmol) and 2-(4-(benzo[d]thiazol-2-yl)phenyl)anthracene-9,10-dione (7.0 g, 16.7 mmol), obtained was the objective compound (136) (6.0 g, overall yield: 20.3%).

¹H NMR (200 MHz, CDCl₃): δ 7.20-7.21 (m, 2H), 7.31-7.33 (m, 8H), 7.47-7.54 (m, 19H), 7.68-7.72 (m, 3H), 7.89-7.91 (d, 1H), 8.11 (d, 1H), 8.21 (d, 1H)

MS/FAB: 692.24 (found), 691.88 (calculated)

### Preparation Example 38

### Preparation of Compound (137)

According to the same procedure as in Preparation Example 1 but using 2-bromobiphenyl (11.7 g, 50.3 mmol) and 2-(4-(benzo[d]thiazol-2-yl)phenyl)anthracene-9,10-dione (7.0 g, 16.7 mmol), obtained was the objective compound (137) (3.8 g, overall yield: 15.3%).

¹H NMR (200 MHz, CDCl₃): δ 7.25-7.31 (m, 8H), 7.46-7.51 (m, 4H), 7.52-7.60 (m, 11H), 7.68-7.73 (m, 3H), 7.87 (d, 1H), 8.12 (d, 1H), 8.23 (d, 1H)

MS/FAB: 691.23 (found), 691.88 (calculated)

### Preparation Example 39

### Preparation of Compound (138)

A reaction vessel was charged with 1,3,5-tribromobenzene (20.0 g, 63.5 mmol), phenyl boronic acid (16.2 g, 133.4 mmol) and trans-dichlorobis(triphenylphosphine)palladium (II) (Pd(PPh₃)₂Cl₂) (4.4 g, 6.3 mmol), and toluene (600 mL) and 2M sodium carbonate (Na₂CO₃) (200 mL), and the mixture was stirred at 90° C. After 4 hours, the temperature was lowered to room temperature, and water (200 mL) was added to quench the reaction. The reaction mixture was extracted with dichloromethane (300 mL), and the extract was dried under reduced pressure. Purification via column chromatography (eluent: n-hexane) gave 1-bromo-3,5-diphenylbenzene (9.6 g, 31.0 mmol).

According to the same procedure as in Preparation Example 1 but using 1-bromo-3,5-diphenylbenzene (9.6 g, 31.0 mmol), 2-(4-(benzo[d]thiazol-2-yl)phenyl)anthracene-9,10-dione (7.0 g, 16.7 mmol), obtained was the objective compound (138) (5.0 g, overall yield: 32.8%).

¹H NMR (200 MHz, CDCl₃): δ 7.19-7.22 (m, 4H), 7.24-7.31 (m, 10H), 7.48-7.58 (m, 15H), 7.62-7.65 (m, 8H), 7.66-7.70 (m, 3H), 7.87 (d, 1H), 8.11 (d, 1H), 8.21 (d, 1H)

MS/FAB: 843.30 (found), 844.07 (calculated)

### Preparation Example 40

### Preparation of Compound (139)

A reaction vessel was charged with 1,2-dibromobenzene (20.0 g, 84.8 mmol), 2-naphthalene boronic acid (16.0 g, 93.3 mmol), and trans-dichlorobis(triphenylphosphine)palladium (II) (Pd(PPh₃)₂Cl₂) (5.9 g, 8.4 mmol), and the mixture was stirred under reflux in 2M sodium carbonate (Na₂CO₃) (150 mL) and toluene (500 mL). After two hours, the reaction mixture was extracted with dichloromethane (500 mL), and the extract was filtered under reduced pressure. Recrystallization from methanol (300 mL) gave 2-(2-bromophenyl)naphthalene (20.0 g, 70.6 mmol).

According to the same procedure as in Preparation Example 1 but using 2-(2-bromophenyl)naphthalene (14.24 g, 50.3 mmol) and 2-(4-(benzo[d]thiazol-2-yl)phenyl)anthracene-9,10-dione (7.0 g, 16.7 mmol), obtained was the objective compound (139) (5.0 g, overall yield: 21.7%).

¹H NMR (200 MHz, CDCl₃): δ 7.22-7.38 (m, 10H), 7.48-7.51 (m, 13H), 7.61-7.67 (m, 6H), 7.70-7.74 (d, 3H), 7.89-7.90 (s, 3H), 8.12 (d, 1H), 8.23 (d, 1H)

MS/FAB: 791.26 (found), 792.0 (calculated)

### Preparation Example 41

### Preparation of Compound (140)

According to the same procedure as in Preparation Example 35 but using 2,7-bromo-9,9-dimethylfluorene (100.0 g, 284.0 mmol), tetrahydrofuran (800 mL), n-buthyllithium (2.5M solution in n-hexane) (124.9 mL, 312.4 mmol), N,N-dimethylformaldehyde (41.5 g, 568.0 mmol) and 2-aminophenol (13.7 g, 109.5 mmol) under nitrogen atmosphere, obtained was 7-(benzo[d]thiazol-2-yl)-9,9-dimethyl-9H-fluoren-2-yl-2-boronic acid (19.0 g, 51.18 mmol).

According to the same procedure as in Preparation Example 35 but using 2-chloroanthraquinone (7.8 g, 32.3 mmol), 7-(benzo[d]thiazol-2-yl)-9,9-dimethyl-9H-fluoren-2-yl-2-boronic acid (18.0 g, 48.4 mmol), tetrakis(triphenylphosphine)palladium (O) (Pd(PPh₃)₄) (3.7 g, 3.2 mmol) and 2M potassium carbonate (K₂CO₃) (150 mL), obtained was 2-(2-benzo[d]thiazol-2-yl)-9,9-dimethyl-9H-fluoren-7-yl)anthracene-9,10-dione (16.3 g, 30.7 mmol).

According to the same procedure as in Preparation Example 1 but using 2-bromonaphthalene (8.15 g, 39.35 mmol) and 2-(2-benzo[d]thiazol-2-yl)-9,9-dimethyl-9H-fluoren-7-yl)anthracene-9,10-dione (7.0 g, 13.1 mmol) under nitrogen atmosphere, obtained was the objective compound (140) (5.2 g, overall yield: 31.5%).

¹H NMR (200 MHz, CDCl₃): δ 1.67 (s, 6H), 7.35-7.39 (m, 6H), 7.51-7.63 (m, 7H), 7.64-7.70 (m, 6H), 7.72-7.81 (m, 5H), 7.91-7.94 (m, 5H), 8.11 (d, 1H), 8.21 (d, 1H)

MS/FAB: 756.27 (found), 755.96 (calculated)

### Preparation Example 42

### Preparation of Compound (141)

According to the same procedure as in Preparation Example 1 but using 2-bromo-9,9-dimethylfluorene (3.58 g, 39.36 mmol) and 2-(2-benzo[d]thiazol-2-yl)-9,9-dimethyl-9H-fluoren-7-yl)anthracene-9,10-dione (7.0 g, 13.12 mmol), obtained was the objective compound (141) (6.64 g, overall yield: 32.5%).

¹H NMR (200 MHz, CDCl₃): δ 1.67 (s, 18H), 7.25-7.38 (m, 6H), 7.51-790 (m, 22H), 8.14 (d, 1H), 8.22 (d, 1H)

MS/FAB: 887.36 (found), 888.17 (calculated)

### Preparation Example 43

### Preparation of Compound (142)

According to the same procedure as in Preparation Example 1 but using 4-bromobiphenyl (9.17 g, 39.3 mmol) and 2-(2-benzo[d]thiazol-2-yl)-9,9-dimethyl-9H-fluoren-7-yl)anthracene-9,10-dione (7.0 g, 13.1 mmol), obtained was the objective compound (142) (5.69 g, overall yield: 36.2%).

¹H NMR (200 MHz, CDCl₃): δ 1.66 (d, 6H), 7.21-7.28 (m, 2H), 7.31-7.37 (m, 6H), 7.47-7.51 (m, 4H), 7.52-7.63 (m, 13H), 7.65-7.69 (m, 2H), 7.72-7.79 (m, 3H, 7.90-7.92 (m, 3H), 8.13 (d, 1H), 8.24 (d, 1H)

MS/FAB: 807.30 (found), 808.04 (calculated)

### Preparation Example 44

### Preparation of Compound (143)

According to the same procedure as in Preparation Example 1 but using 2-bromobiphenyl (9.17 g, 39.36 mmol) and 2-(2-benzo[d]thiazol-2-yl)-9,9-dimethyl-9H-fluoren-7-yl)anthracene-9,10-dione (7.0 g, 13.1 mmol), obtained was the objective compound (143) (3.63 g, overall yield: 23.9%).

¹H NMR (200 MHz, CDCl₃): δ 1.64 (s, 6H), 7.19-7.34 (m, 12H), 7.49-7.51 (d, 4H), 7.52-7.62 (m, 9H), 7.68-7.72 (m, 3H), 7.73-7.78 (m, 2H0, 7.88-7.91 (m, 3H), 8.11 (d, 1H), 8.25 (d, 1H)

MS/FAB: 807.30 (found), 808.04 (calculated)

### Preparation Example 45

### Preparation of Compound (144)

According to the same procedure as in Preparation Example 1 but using 1-bromo-3,5-diphenylbenzene (12.1 g, 39.3 mmol) and 2-(2-benzo[d]thiazol-2-yl)-9,9-dimethyl-9H-fluoren-7-yl)anthracene-9,10-dione (7.0 g, 13.12 mmol), obtained was the objective compound (144) (7.42 g, overall yield: 43.3%).

¹H NMR (200 MHz, CDCl₃): δ 1.66 (s, 6H), 7.21-7.27 (m, 4H0, 7.29-7.34 (m, 10H), 7.48-7.50 (d, 8H), 7.51-7.64 (m, 5H), 7.65-7.71 (m, 8H), 7.71-7.76 (m, 3H), 7.89-7.91 (m, 3H), 8.13 (d. 1H), 8.24 (d, 1H)

MS/FAB: 959.36 (found), 960.23 (calculated)

### Preparation Example 46

### Preparation of Compound (145)

According to the same procedure as in Preparation Example 1 but using 2-(2-bromophenyl)naphthalene (11.15 g, 39.36 mmol) and 2-(2-benzo[d]thiazol-2-yl)-9,9-dimethyl-9H-fluoren-7-yl)anthracene-9,10-dione (7.0 g, 13.12 mmol), obtained was the objective compound (145) (3.82 g, overall yield: 23.5%).

¹H NMR (200 MHz, CDCl₃): δ 1.65 (d, 6H), 7.22-7.30 (m, 10H), 7.51-7.65 (m, 11H), 7.67-7.78 (m, 11H), 7.89-7.92 (m, 5H), 8.12 (d, 1H0, 8.22 (d, 1H)

MS/FAB: 907.23 (found), 908.16 (calculated)

### Preparation Example 47

### Preparation of Compound (146)

According to the same procedure as in Preparation Example 35 but using 2,6-dibromonaphthalene (100.0 g, 349.69 mmol), n-buthyllithium (2.5M solution in n-hexane) (153.87 mL, 384.67 mmol), N,N-dimethylformaldehyde (51.13 g, 699.38 mmol), 2-aminophenol (17.57 g, 140.38 mmol) and trimethylborate (18.36 g, 176.34 mmol) under nitrogen obtained was 6-(benzo[d]thiazol-2-yl)naphthalen-2-yl-2-boronic acid (24.62 g, 80.68 mmol).

According to the same procedure as in Preparation Example 35 but using 2-chloroanthraquinone (10.0 g, 41.21 mmol), 6-(benzo[d]thiazol-2-yl) naphthalen-2-yl-2-boronic acid (15.09 g, 49.45 mmol), tetrakis palladium (II) triphenylphosphine (Pd(PPh₃)₄) (4.76 g, 4.12 mmol) and 2M potassium carbonate (K₂CO₃) (200 mL) in ethylene glycol dimethyl ether (DME) (500 mL) and ethanol (200 mL), obtained was 2-(2-benzo[d]thiazol-2-yl)naphthalen-6-yl)anthracene-9,10-dione (18.11 g, 38.74 mmol).

According to the same procedure as in Preparation Example 1 but using 2-bromonaphthalene (13.29 g, 64.17 mmol) and 2-(2-benzo[d]thiazol-2-yl)naphthalen-6-yl)anthracene-9,10-dione (10.0 g, 21.39 mmol) under nitrogen atmosphere, obtained was the objective compound (146) (8.02 g, overall yield: 36.2%).

¹H NMR (200 MHz, CDCl₃): δ 7.30-7.32 (m, 4H), 7.34-7.36 (d, 2H), 7.53-7.56 (m, 7H), 7.64-7.67 (d, 6H), 7.71-7.73 (m, 5H), 7.86-7.88 (S, 5H), 8.12 (d, 1H), 8.21 (d, 1H)

MS/FAB: 689.22 (found), 689.86 (calculated)

### Preparation Example 48

### Preparation of Compound (147)

According to the same procedure as in Preparation Example 1 but using 2-bromo-9,9-dimethylfluorene (17.53 g, 64.17 mmol) and 2-(2-benzo[d]thiazol-2-yl)naphthalen-6-yl)anthracene-9,10-dione (10.0 g, 21.39 mmol), obtained was the objective compound (147) (9.06 g, overall yield: 21.3%).

¹H NMR (200 MHz, CDCl₃): δ 1.66 (S, 12H), 7.31-7.37 (m, 6H), 7.51-7.56 (m, 8H), 7.62-7.37 (m, 4H), 7.73-7.77 (m, 4H), 7.83-7.91 (m, 6H), 8.06 (s, 1H), 8.11 (d, 1H) 8.22 (d, 1H)

MS/FAB: 821.31 (found), 822.07 (calculated)

### Preparation Example 49

### Preparation of Compound (148)

According to the same procedure as in Preparation Example 1 but using 4-bromobiphenyl (14.94 g, 64.08 mmol) and 2-(2-benzo[d]thiazol-2-yl)naphthalen-6-yl)anthracene-9,10-dione (10.0 g, 21.39 mmol), obtained was the objective compound (148) (7.79 g, overall yield: 52.5%).

¹H NMR (200 MHz, CDCl₃): δ 7.21-7.23 (m, 2H), 7.30-7.33 (m, 6H), 7.46-7.49 (m, 4H), 7.51-7.54 (m, 12H), 7.67-7.66 (m, 2H), 7.72-7.74 (d, 3H), 8.12 (d, 1H), 8.23 (d, 1H)

MS/FAB: 741.25 (found), 741.94 (calculated)

### Preparation Example 50

### Preparation of Compound (149)

According to the same procedure as in Preparation Example 35 but using 1,4-dibromonaphthalene (100.0 g, 349.69 mmol), n-buthyllithium (2.5M solution in n-hexane) (153.87 mL, 384.67 mmol), N,N-dimethylformaldehyde (51.13 g, 699.38 mmol) and 2-aminophenol (17.57 g, 140.38 mmol) under nitrogen atmosphere, obtained was 2-(1-bromonaphthalen-4-yl)benzo[d]thiazole (31.26 g, 91.89 mmol).

According to the same procedure as in Preparation Example 35 but using 2-(1-bromonaphthalen-4-yl)benzo[d]thiazole (30.0 g, 88.17 mmol), n-buthyllithium (2.5M solution in n-hexane) (38.79 mL, 96.99 mmol) and trimethylborate (18.36 g, 176.34 mmol) under nitrogen atmosphere, obtained was 4-(benzo[d]thiazol-2-yl)naphthalen-1-yl-1-boronic acid (18.57 g, 80.68 mmol).

According to the same procedure as in Preparation Example 35 but using 2-chloroanthraquinone (10.0 g, 41.21 mmol), 4-(benzo[d]thiazol-2-yl)naphthalen-1-yl-1-boronic acid (15.09 g, 49.45 mmol), tetrakis(triphenylphosphine)palladium (O) (Pd(PPh₃)₄) (4.76 g, 4.12 mmol) and 2M potassium carbonate (K₂CO₃) (200 mL) dissolved in ethylene glycol dimethyl ether (DME) (500 mL) and ethanol (200 mL), obtained was 2-(1-benzo[d]thiazol-2-yl)naphthalen-4-yl)anthracene-9,10-dione (16.74 g, 35.81 mmol).

According to the same procedure as in Preparation Example 1 but using 2-bromonaphthalene (13.29 g, 64.17 and 2-(1-benzo[d]thiazol-2-yl)naphthalen-4-yl)anthracene-9,10-dione (10.0 g, 21.39 mmol) under nitrogen atmosphere, obtained was the objective compound (149) (6.65 g, overall yield: 26.5%).

¹H NMR (200 MHz, CDCl₃): δ 7.31-7.34 (m, 8H), 7.55-7.58 (m, 5H), 7.61-7.64 (m, 2H), 7.67-7.70 (m, 8H), 7.73-7.75 (m, 3H), 7.87-7.89 (s, 3H), 8.11 (d, 1H), 8.21 (d, 1H)

MS/FAB: 690.22 (found), 689.86 (calculated)

### Preparation Example 51

### Preparation of Compound (150)

According to the same procedure as in Preparation Example 35 but using 4,4-dibromobiphenyl (100.0 g, 320.51 mmol), n-buthyllithium (2.5M solution in n-hexane) (141.03 mL, 352.56 mmol), N,N-dimethylformaldehyde (46.86 g, 641.02 mmol), 2-aminophenol (15.82 g, 126.38 mmol), n-buthyllithium (2.5M solution in n-hexane) (36.04 mL, 90.09 mmol) and trimethylborate (12.796 g, 122.86 mmol) under nitrogen atmosphere, obtained was 4 (benzo[d]thiazol-2-yl)biphenyl-4-yl-4′-boronic acid (20.07 g, 80.68 mmol).

According to the same procedure as in Preparation Example 35 but using 2-chloroanthraquinone (10.0 g, 41.21 mmol), 4-(benzo[d]thiazol-2-yl)biphenyl-4-yl-4-boronic acid (20.47 g, 61.82 mmol), tetrakis(triphenylphosphine)palladium (O) (Pd(PPh₃)₄) (4.76 g, 4.12 mmol) and 2M potassium carbonate (K₂CO₃) (200 mL) dissolved in ethylene glycol dimethyl ether (DME) (500 mL) and ethanol (200 mL), obtained was 2-(4-benzo[d]thiazol-2-yl)biphenyl-4-yl)anthracene-9,10-dione (17.82 g, 36.09 mmol).

According to the same procedure as in Preparation Example 1 but using 2-bromonaphthalene (12.59 g, 60.78 and 2-(4-benzo[d]thiazol-2-yl)biphenyl-4-yl)anthracene-9,10-dione (10.0 g, 20.26 mmol) under nitrogen atmosphere, obtained was the objective compound (150) (6.03 g, 49.8%).

¹H NMR (200 MHz, CDCl₃): δ 7.30-7.32 (m, 6H), 7.49-7.52 (m, 13H), 7.65-7.67 (m, 6H), 7.71-7.72 (d, 3H), 7.91-7.92 (d, 3H), 8.13 (d, 1H), 8.23 (d, 1H)

MS/FAB: 716.24 (found), 715.9 (calculated)

### Preparation Example 52

Under nitrogen atmosphere, an excess amount of 10N potassium hydroxide (KOH) solution (24.1 g, 430 mmol) was added to 5-amino-6-methylbenzothiazole (20.0 g, 143.68 mmol), and ethylene glycol solvent (20 mL) was added thereto. After stirring the mixture at 125° C. for 15 hours, the temperature was lowered to room temperature, and conc. hydrochloric acid (HCl) (5.0 g) was added to the reaction mixture. The reaction was quenched by extraction with ethyl acetate (500 mL) and washing with water (1000 mL). Purification via column chromatography (eluent: dichloromethane/n-hexane) gave 2-amino-4-methylbenzenethiazole (17.78 g, 127.73 mmol).

According to the same procedure as in Preparation Example 35 but using 2-amino-4-methylbenzethiazole (20.0 g, 143.68 mmol), 4-bromobenzaldehyde (26.58 g, 143.68 mmol) and n-buthyllithium (2.5M solution in n-hexane) (21.04 mL, 52.6 mmol) and trimethylborate (10.31 g, 98.63 mmol), obtained was 4-(6-methylbenzo[d]thiazol-2-yl)phenylboronic acid (15.27 g, 56.74 mmol).

According to the same procedure as in Preparation Example 35 but using 2-chloroanthraquinone (10.0 g, 41.21 mmol), 4-(6-methylbenzo[d]thiazol-2-yl)phenylboronic acid (14.42 g, 53.57 mmol), tetrakis(triphenylphosphine)palladium (O) (Pd(PPh₃)₄) (4.76 g, 4.12 mmol) and 2M potassium carbonate (K₂CO₃) (200 mL) dissolved in ethylene glycol dimethyl ether (DME) (500 mL) and ethanol (200 mL), obtained was 2-(4-(6-methylbenzo[d]thiazol-2-yl)phenyl)anthracene-9,10-dione (12.25 g, 38.33 mmol).

According to the same procedure as in Preparation Example 1 but using 2-bromonaphthalene (14.39 g, 69.52 mmol) and 2-(4-(6-methylbenzo[d]thiazol-2-yl)phenyl)anthracene-9,10-dione (10.0 g, 23.17 mmol) under nitrogen atmosphere, obtained was the objective compound (151) (7.45 g, overall yield: 32.1%).

¹H NMR (200 MHz, CDCl₃): δ 2.35 (s, 3H), 7.29-7.34 (m, 7H), 7.54-7.57 (m, 7H), 7.69-7.71 (m, 6H), 7.73-7.75 (m, 3H), 7.89-7.91 (m, 4H), 8.12 (d, 1H)

MS/FAB: 716.24 (found), 715.9 (calculated)

### Preparation Example 53

### Preparation of Compound (152)

According to the same procedure as in Preparation Example 52 but using 2-amino-6-bromobenzothiazole (15.0 g, 65.47 mmol) and 10N potassium hydroxide (KOH) (10 g, 180 mmol), obtained was 2-amino-5-bromobenzenethiol (11.6 g, 56.84 mmol).

A solution of 2-amino-5-bromobenzenethiol (10.0 g, 48.99 mmol), phenylboronic acid (7.15 g, 58.68 mmol) and tetrakis(triphenylphosphine)palladium (O) (Pd(PPh₃)₄) (5.65 g, 4.89 mmol) dissolved in 2M sodium carbonate (Na₂CO₃) (150 mL), ethylene glycol dimethyl ether (DME) (500 mL) and ethanol (200 mL) was stirred under reflux for 20 hours. After cooling to room temperature, water (300 mL) was added to the reaction mixture, and the resultant mixture was stirred and extracted with ethyl acetate (600 mL). The extract was distilled under reduced pressure, and recrystallized from n-hexane (300 mL) to obtain 2-amino-5-phenylbenzenethiol (8.77 g, 43.45 mmol).

According to the same procedure as in Preparation Example 35 but using 2-amino-5-phenylbenzenethiol (8.77 g, 43.35 mmol), 4-bromobenzaldehyde (8.04 g, 43.45 mmol), dimethylsulfoxide (DMSO) (40 mL), n-butyllithium (2.5M solution in n-hexane) (8.74 mL, 21.84 mmol), trimethylborate (3.69 g, 35.49 mmol), obtained was 4-(6-phenylbenzo[d]thiazol-2-yl)phenylboronic acid (6.06 g, 18.29 mmol).

According to the same procedure as in Preparation Example 35 but using 2-chloroanthraquinone (10.0 g, 41.21 mmol), 4-(6-phenylbenzo[d]thiazol-2-yl)phenylboronic acid (20.47 g, 61.82 mmol) and tetrakis(triphenylphosphine)palladium (O) (Pd(PPh₃)₄) (4.76 g, 4.12 mmol) dissolved in 2M potassium carbonate (K₂CO₃) (200 mL), ethylene glycol dimethyl ether (DME) (500 mL) and ethanol (200 mL), obtained was 2-(4-(6-phenylbenzo[d]thiazol-2-yl)phenyl)anthracene-9,10-dione (18.92 g, 38.33 mmol).

According to the same procedure as in Preparation Example 1 but using 2-bromonaphthalene (12.59 g, 60.78 mmol) and 2-(4-(6-phenylbenzo[d]thiazol-2-yl)phenyl)anthracene-9,10-dione (10.0 g, 20.26 mmol) under nitrogen atmosphere, obtained was the objective compound (152) (6.88 g, overall yield: 51.7%).

¹H NMR (200 MHz, CDCl₃): δ 7.24-7.26 (m, 1H), 7.30-7.34 (m, 8H), 7.49-7.51 (m, 2H), 7.51-7.52 (m, 7H), 7.69-7.72 (m, 6H), 7.72-7.84 (m, 4H), 7.90-7.92 (S, 3H), 8.29 (d, 1H), 8.34 (d, 1H)

MS/FAB: 716.24 (found), 715.9 (calculated)

### Preparation Example 54

### Preparation of Compound (153)

According to the same procedure as in Preparation Example 35 but using 2-bromobenzaldehyde (59.10 g, 319.51 mmol), 2-aminophenol (40.0 g, 319.51 mmol), dimethylsulfoxide (400 mL), n-butyllithium (2.5M solution in n-hexane) (24.81 mL, 62.03 mmol) and trimethylborate (10.76 g, 103.35 mmol), obtained was 2-(benzo[d]thiazol-2-yl)phenylboronic acid (8.3 g, 32.53 mmol).

According to the same procedure as in Preparation Example 35 but using 2-chloroanthraquinone (5.26 g, 21.69 mmol), 2-(benzo[d]thiazol-2-yl)phenylboronic acid (8.3 g, 32.53 mmol) and tetrakis(triphenylphosphine)palladium (O) (Pd(PPh₃)₄) (2.51 g, 2.17 mmol) dissolved in 2M potassium carbonate (K₂CO₃) (80 mL), ethylene glycol dimethyl ether (DME) (200 mL) and ethanol (800 mL), obtained was 2-(2-benzo[d]thiazol-2-yl)phenyl)anthracene-9,10-dione (8.69 g, 20.82 mmol).

According to the same procedure as in Preparation Example 1 but using 2-bromonaphthalene (19.83 g, 95.8 mmol) and 2-(2-benzo[d]thiazol-2-yl)phenyl)anthracene-9,10-dione (10.0 g, 23.95 mmol) under nitrogen atmosphere, obtained was the objective compound (153) (6.50 g, 10.17 mmol, overall yield: 51.4%).

¹H NMR (200 MHz, CDCl₃): δ 7.25-7.32 (m, 8H), 7.51-7.58 (m, 7H), 7.62-7.66 (m, 6H), 7.66-7.73 (m, 3H), 8.91-7.92 (s, 3H), 8.15 (d, 1H), 8.25 (d, 1H)

MS/FAB: 639.21 (found), 639.80 (calculated)

### Preparation Example 55

### Preparation of Compound (154)

According to the same procedure as in Preparation Example 35 but using 3-bromobenzaldehyde (59.10 g, 319.51 mmol), 2-aminophenol (40.0 g, 319.51 mmol), n-butyllithium (2.5M solution in n-hexane) (24.81 mL, 62.03 mmol) and trimethylborate (10.76 g, 103.35 mmol), obtained was 3-(benzo[d]thiazol-2-yl)phenylboronic acid (12.61 g, 49.42 mmol).

According to the same procedure as in Preparation Example 35 but using 2-chloroanthraquinone (7.99 g, 32.95 mmol), 3-(benzo[d]thiazol-2-yl)phenylboronic acid (12.61 g, 49.42 mmol) and tetrakis(triphenylphosphine)palladium (O) (Pd(PPh₃)₄) (3.80 g, 3.29 mmol) dissolved in 2M ethylene glycol dimethyl ether (DME) (300 mL) and ethanol (100 mL), obtained was 2-(3-benzo[d]thiazol-2-yl)phenyl)anthracene-9,10-dione (11.17 g, 26.76 mmol).

According to the same procedure as in Preparation Example 1 but using 2-bromonaphthalene (19.83 g, 95.8 mmol) and 2-(3-benzo[d]thiazol-2-yl)phenyl)anthracene-9,10-dione (10.0 g, 23.95 mmol) under nitrogen atmosphere, obtained was the objective compound (154) (7.61 g, 11.89 mmol, overall yield: 55.4%).

¹H NMR (200 MHz, CDCl₃): δ 7.29-7.47 (m, 9H), 7.48-7.57 (m, 5H), 7.61-7.72 (m, 10H), 7.88-7.91 (s, 3H), 8.09-7.12 (m, 1H), 8.18-8.22 (m, 1H)

MS/FAB: 640.21 (found), 639.80 (calculated)

### Preparation Example 56

### Preparation of Compound (155)

A reaction vessel was charged with copper bromide (101.0 g, 0.45 mmol), tert-butyl nitrate (58.34 mL, 0.49 mmol) and acetonitrile (800 mL), and the mixture was stirred at 70° C. After 1 hour, 2,6-diaminoanthraquinone (45.0 g, 0.19 mmol) was added thereto, and the resultant mixture was stirred at 85° C. After 48 hours, 20% hydrochloric acid (1 L) was added thereto, and the mixture was stirred for 1 hours. The precipitate produced was filtered and washed several times with water and methanol. Washing again with acetone and dichloromethane twice each gave 2,6-dibromoanthracene-9,10-dione (50.0 g, 54.65 mmol).

A solution of 2,6-dibromoanthracene-9,10-dione (10.0 g, 27.32 mmol), 4-benzo[d]thiazol-2-yl)phenylboronic acid (17.42 g, 68.31 mmol) and tetrakis(triphenylphosphine)palladium (O) (Pd(PPh₃)₄) (3.15 g, 2.73 mmol) dissolved in 2M potassium carbonate (K₂CO₃) (100 mL), ethylene glycol dimethyl ether (DME) (300 mL) and ethanol (100 mL) was stirred under reflux for 20 hours. After cooling to room temperature, water (200 mL) was added to the reaction mixture, and the resultant mixture was stirred, and extracted with ethyl acetate solvent (300 mL). The extract was dried under reduced pressure, and recrystallized from n-hexane (300 mL) to obtain 2,6-bis(4-benzo[d]thiazol-2-yl)phenyl)anthracene-9,10-dione (14.79 g, 23.60 mmol).

According to the same procedure as in Preparation Example 1 but using 2-bromonaphthalene (9.91 g, 47.88 mmol) and 2,6-bis(4-benzo[d]thiazol-2-yl)phenyl)anthracene-9,10-dione (10.0 g, 15.96 mmol) under nitrogen atmosphere, obtained was the objective compound (155) (8.0 g, 9.18 mmol, overall yield: 62.4%).

¹H NMR (200 MHz, CDCl₃): δ 7.25-7.31 (m, 4H), 7.50-7.56 (m, 16H), 7.62-7.69 (m, 4H), 7.70-7.72 (d, 4H), 7.89-7.91 (s, 4H), 8.08-8.12 (m, 2H), 8.20-8.23 (m, 2H)

MS/FAB: 848.23 (found), 849.07 (calculated)

### Preparation Example 57

### Preparation of Compound (156)

According to the same procedure as in Preparation Example 1 but using 2-bromo-9,9-dimethylfluorene (13.08 g, 47.87 mmol), 2,6-bis(4-benzo[d]thiazol-2-yl)phenyl)anthracene-9,10-dione (10.0 g, 15.96 mmol), obtained was the objective compound (156) (7.01 g, 54.7%).

¹H NMR (200 MHz, CDCl₃): δ 1.68 (s, 12H), 7.24-7.29 (t, 2H), 7.31-7.39 (t, 2H), 7.50-7.64 (m, 18H), 7.72-7.78 (m, 4H), 7.80-7.82 (d, 2H), 7.87-7.90 (m, 4H), 8.11-8.13 (m, 2H), 8.19-8.21 (m, 2H)

MS/FAB: 982.33 (found), 981.27 (calculated)

### Preparation Example 58

### Preparation of Compound (157)

According to the same procedure as in Preparation Example 1 but using 4-bromobiphenyl (11.16 g, 47.87 mmol), 2,6-bis(4-benzo[d]thiazol-2-yl)phenyl)anthracene-9,10-dione (10.0 g, 15.96 mmol), obtained was the objective compound (157) (6.59 g, 54.9%).

¹H NMR (200 MHz, CDCl₃): δ 7.16-7.21 (m, 2H), 7.23-7.31 (m, 4H), 7.45-7.50 (m, 4H), 7.50-7.57 (s, 22H), 7.71-7.73 (d, 2H), 8.89-8.90 (s, 2H), 8.07-8.12 (m, 2H), 8.19-8.23 (m, 2H)

MS/FAB: 902.27 (found), 901.15 (calculated)

### Preparation Example 59

### Preparation of Compound (158)

According to the same procedure as in Preparation Example 56 but using 2,6-dibromoanthracene-9,10-dione (10.0 g, 27.32 mmol), 6-(benzo[d]thiazol-2-yl)naphthalen-2-yl-2-boronic acid (20.0 g, 54.64 mmol) and tetrakis(triphenylphosphine)palladium (O) (Pd(PPh₃)₄) (6.31 g, 5.46 mmol) dissolved in 2M potassium carbonate (K₂CO₃) (200 mL), ethylene glycol dimethyl ether (DME) (500 mL) and ethanol (200 mL), obtained was 2,6-bis(2-benzo[d]thiazol-2-yl)naphthalen-6-yl)anthracene-9,10-dione (28.83 g, 39.67 mmol).

According to the same procedure as in Preparation Example 1 but using 2-bromonaphthalene (8.55 g, 41.27 mmol) and 2,6-bis(2-benzo[d]thiazol-2-yl)naphthalen-6-yl)anthracene-9,10-dione (10.0 g, 13.76 mmol) under nitrogen atmosphere, obtained was the objective compound (158) (6.05 g, 6.37 mmol, overall yield: 67.4%).

¹H NMR (200 MHz, CDCl₃): δ 7.25-7.32 (m, 4H), 7.51-7.58 (m, 12H), 7.62-7.68 (m, 4H0, 7.69-7.72 (m, 8H), 7.88-7.90 (s, 8H), 8.12-8.14 (m, 2H), 8.23-8.25 (m, 2H)

MS/FAB: 848.23 (found), 849.07 (calculated)

### Preparation Example 60

### Preparation of Compound (159)

According to the same procedure as in Preparation Example 1 but using 2-bromo-9,9-dimethylfluorene (11.27 g, 41.27 mmol) and 2,6-bis(2-benzo[d]thiazol-2-yl)naphthalen-6-yl)anthracene-9,10-dione (10.0 g, 13.76 mmol), obtained was the objective compound (159) (6.09 g, 57.4%).

¹H NMR (200 MHz, CDCl₃): δ 1.65 (s, 12H), 7.21-7.28 (t, 2H), 7.30-7.37 (t, 2H), 7.50-7.61 (m, 14H), 7.70-7.78 (m, 8H), 7.82-7.91 (m, 10H), 8.08-8.10 (m, 2H0, 8.22-8.24 (m, 2H)

MS/FAB: 1080.36 (found), 1081.39 (calculated)

### Preparation Example 61

### Preparation of Compound (160)

According to the same procedure as in Preparation Example 1 but using 4-bromobiphenyl (11.16 g, 47.87 mmol) and 2,6-bis(2-benzo[d]thiazol-2-yl)naphthalen-6-yl)anthracene-9,10-dione (10.0 g, 13.76 mmol), obtained was the objective compound (160) (6.59 g, 54.9%).

¹H NMR (200 MHz, CDCl₃): δ 7.14-7.22 (m, 2H), 7.24-7.32 (m, 4H), 7.47-7.49 (m, 4H), 7.49-7.56 (m, 18H), 7.71-7.76 (d, 6H), 7.88-7.90 (s, 6H), 8.11-8.13 (m, 2H), 8.24-8.26 (m, 2H)

MS/FAB: 902.27 (found), 901.15 (calculated)

### Example 1-61

### Manufacture of OLED's Using the Compounds According to the Present Invention

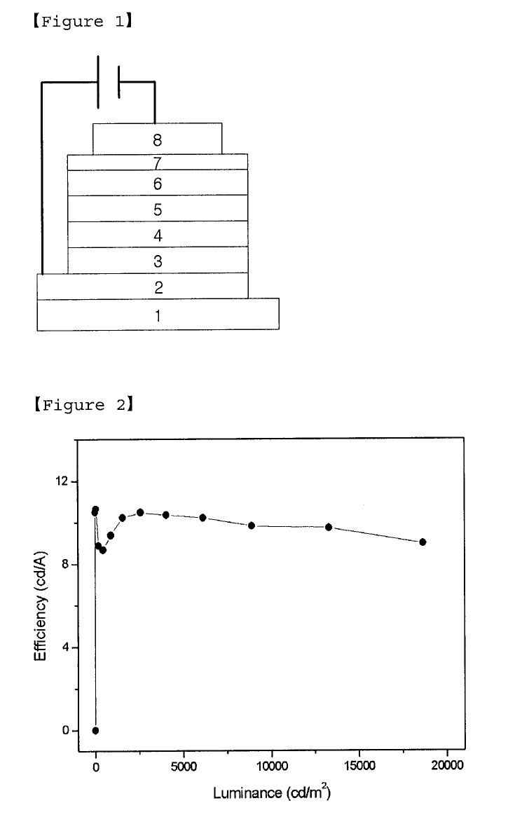

OLED's were manufactured as illustrated in FIG. 1 by using the electron transportation materials according to the present invention.

First, a transparent electrode ITO thin film (2) (15Ω/□) obtained from glass (1) for OLED was subjected to ultrasonic washing with trichloroethylene, acetone, ethanol and distilled water, subsequently, and stored in isopronanol before use.

Then, an ITO substrate was equipped in a substrate folder of a vacuum vapor-deposit device, and 4,4′,4″-tris(N,N-(2-naphthyl)-phenylamino)triphenylamine (2-TNATA, having the structure shown below) was placed in a cell of the vacuum vapor-deposit device, which was then vented to reach 10⁻⁶ torr of vacuum in the chamber. Electric current was applied to the cell to evaporate 2-TNATA to vapor-deposit a hole injection layer (3) with 60 nm of thickness on the ITO substrate.

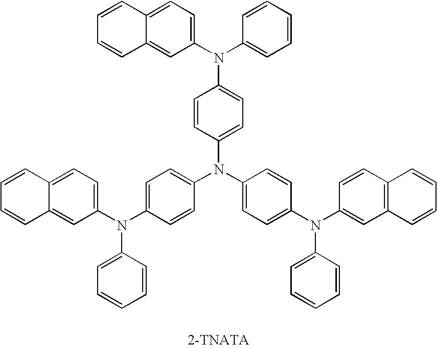

Then, another cell of the vacuum vapor-deposit device was charged with N,N′-bis(α-naphthyl)-N,N′-diphenyl-4,4′-diamine (NPB), and electric current was applied to the cell to evaporate NPB to vapor-deposit a hole transportation layer (4) with 20 nm of thickness on the hole injection layer.

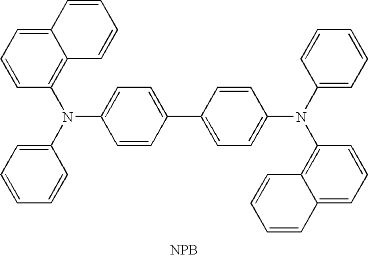

After formation of the hole injection layer and hole transportation layer, an electroluminescent layer was vapor-deposited as follows. One cell of the vacuum deposition device was charged with tris(8-hydroxyquinoline)aluminum (III) (Alq) as an electroluminescent host material, while another cell of said device was charged with coumarin 545T (C545T), respectively. Two substances were doped by evaporating with different rates to vapor-deposit an electroluminescent layer (5) with a thickness of 30 nm on the hole transportation layer. The doping concentration was preferably 2 to 5 mol % on the basis of Alq.

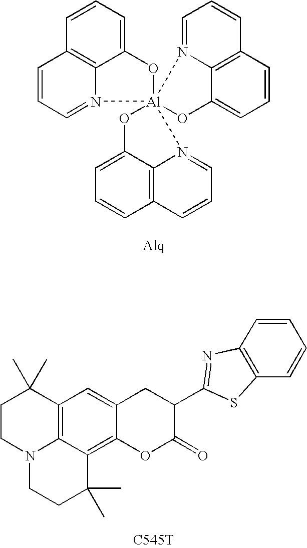

Then, one of the compounds prepared according to Preparation Examples 1 to 61 (for example, Compound 109) was vapor-deposited with a thickness of 20 nm, as an electron transportation layer (6), followed by lithium quinolate (Liq) with a thickness of from 1 to 2 nm as an electron injection layer (7). Thereafter, an Al cathode (8) was vapor-deposited with a thickness of 150 nm by using another vacuum vapor-deposit device to manufacture an OLED.

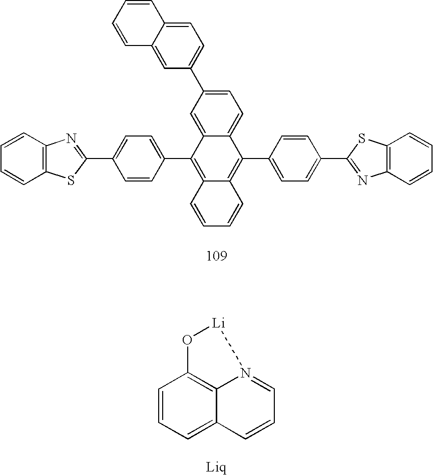

### Comparative Example 1

### Manufacture of an OLED Using Conventional EL Material

A hole injection layer (3), a hole transportation layer (4) and an electroluminescent layer (5) were formed according to the same procedure as described in Example 1 to 61, and Alq (tris(8-hydroxyquinoline)-aluminum (III) having the structure shown below was vapor-deposited with 20 nm of thickness as an electron transportation layer (6), followed by lithium quinolate (Liq) with 1˜2 nm of thickness as an electron injection layer (7). An Al cathode (8) was vapor-deposited by using another vacuum vapor-deposit device with a thickness of 150 nm, to manufacture an OLED.

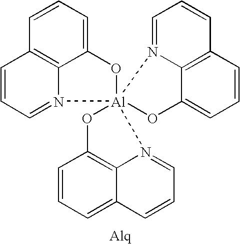

### Experimental Example 1

### Examination of Properties of OLED

Current luminous efficiencies and power efficiencies of OLED's comprising the thiazole system organic electroluminescent compound according to the present invention prepared from Example 1 to 61 and the conventional electroluminescent compound were measured at 1000 cd/m², of which the results are shown in Table 1.

TABLE 1

| | | Operation | Luminous | Power | |
|---|---|---|---|---|---|
| | Electron | voltage | efficiency | efficiency | |
| | transportation | (V) | (cd/A) | (lm/W) | Color |
| | layer | @1000 | @1000 | @1000 | coordinate |
| | material | cd/m² | cd/m² | cd/m² | (x, y) |
| Ex. 1 | Comp. 100 | 5 | 11.7 | 7.3 | 0.29, 0.65 |
| Ex. 10 | Comp. 109 | 4 | 13.1 | 10.3 | 0.29, 0.65 |
| Ex. 12 | Comp. 111 | 4 | 12.2 | 9.6 | 0.28, 0.64 |
| Ex. 19 | Comp. 118 | 4.5 | 12.8 | 8.9 | 0.28, 0.64 |
| Ex. 22 | Comp. 121 | 4 | 12.5 | 9.8 | 0.28, 0.64 |
| Ex. 26 | Comp. 125 | 3.5 | 11.0 | 9.9 | 0.30, 0.65 |
| Ex. 31 | Comp. 130 | 4 | 12.7 | 9.9 | 0.29, 0.65 |
| Ex. 35 | Comp. 134 | 4.5 | 13 | 9.1 | 0.29, 0.64 |
| Ex. 39 | Comp. 138 | 4.5 | 12.9 | 9.0 | 0.29, 0.64 |
| Ex. 42 | Comp. 141 | 4.5 | 13.1 | 9.1 | 0.28, 0.64 |
| Ex. 46 | Comp. 145 | 4.5 | 13.4 | 9.3 | 0.28, 0.64 |
| Ex. 54 | Comp. 153 | 4 | 12.8 | 10.0 | 0.28, 0.65 |
| Ex. 57 | Comp. 156 | 4 | 12.0 | 9.4 | 0.30, 0.64 |
| Comp. | Alq₃ | 6 | 11.6 | 6.1 | 0.30, 0.65 |
| Ex. 1 | | | | | |

As can be seen from Table 1, Compound (109) as the electron transportation material (Example 10) showed highest power efficiency. In particular, Compound (109) of Example 10 and Compound (153) of Example 54 showed about 70% enhancement of power efficiency as compared to the conventional material, Alq, as the electron transportation layer.

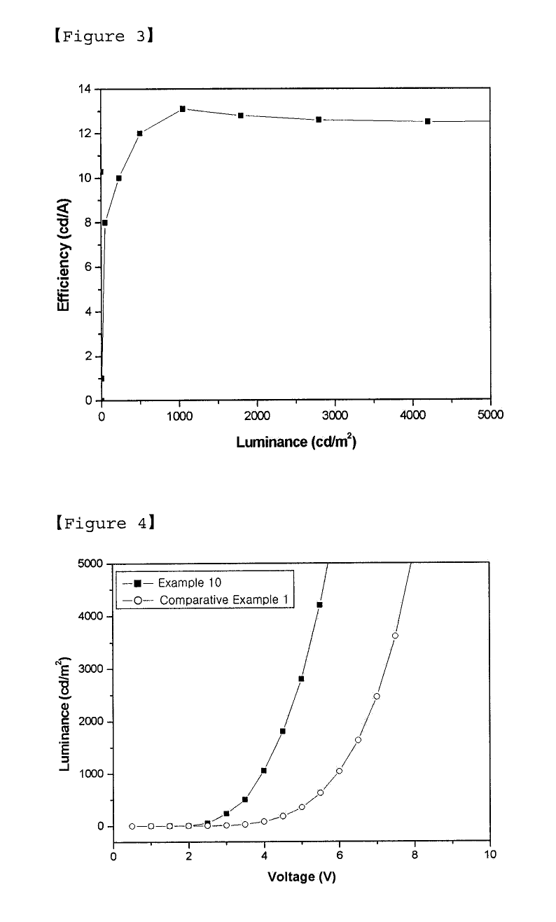

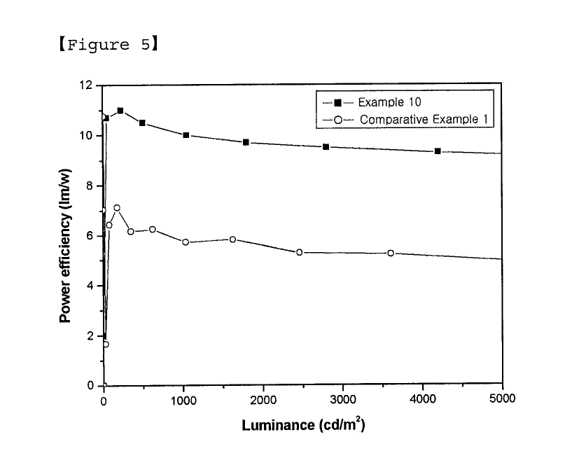

FIG. 2 is a luminous efficiency curve of the conventional electroluminescent material, Alq:C545T, while FIG. 3 is a luminous efficiency curve of Compound (109) employed as the electron transportation material. FIG. 4 and FIG. 5 are luminance-voltage and power efficiency-luminance curves, respectively, which compare Compound (109) according to the present invention and Alq employed as the electron transportation layer.

From Table 1 showing the properties of the compounds developed by the present invention employed as an electron transportation layer, it is confirmed that the compounds developed by the present invention show excellent properties as compared to conventional substances in view of the performances.

It is analyzed that this results come from appropriate combination of thiazole system functional group and anthracene skeletal as new concept of the present invention. The thiazole system functional group comprises heteroatoms such as N and S, so that electron density of the aromatic ring is reduced to give excellent electron transportation property. In addition, anthracene is bipolar in its property, thereby maximizing the ability of carrier delivery.

The combination of both properties as mentioned above is an inherent concept which is looking to the enhancement of carrier delivery property and the role of a host, not only directing to improvement of electroluminescent property of the molecule itself. Consistently, good electroluminescent properties were confirmed in an OLED according to the present invention.

In the meanwhile, in addition to the concept as described above, the present invention designed a molecular structure to have highest electric properties as organic semiconductor in thin film by appropriately combining the position of functional groups, steric hindrance, or the like. It is found that these results contribute to improvement of ability of electron transportation in the present invention.

Particularly, it is found that the improvement of power consumption due to lowered operation voltage in an OLED employing the material according to the invention comes from improvement of current properties, not from simple improvement of luminous efficiency.

### INDUSTRIAL APPLICABILITY

The compounds according to the present invention for an electron transporation layer are advantageous in that they can substantially improve the power efficiency by noticeably lowering the operational voltage and increasing the current efficiency. Thus, it is expected that the material can greatly contribute to reduce the power consumption of an OLED.

1. A thiazole system organic electroluminescent compound represented by Chemical Formula (1): 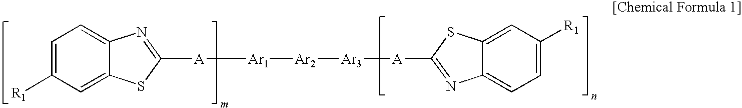
wherein, A is a chemical bond or 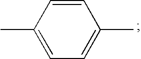
if m is 0, Ar₁ is hydrogen, phenyl, 1-naphthyl or 2-naphthyl;
if m is 1 or 2, Ar₁ is selected from following structures; 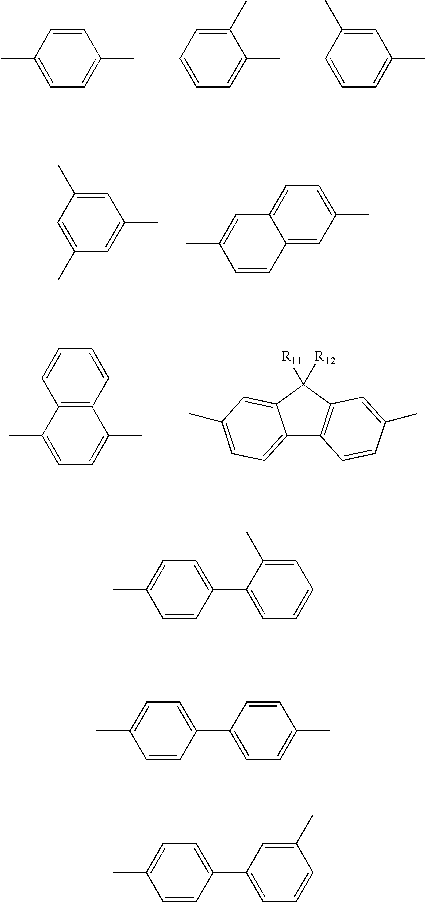
Ar₂ is selected from following structures; 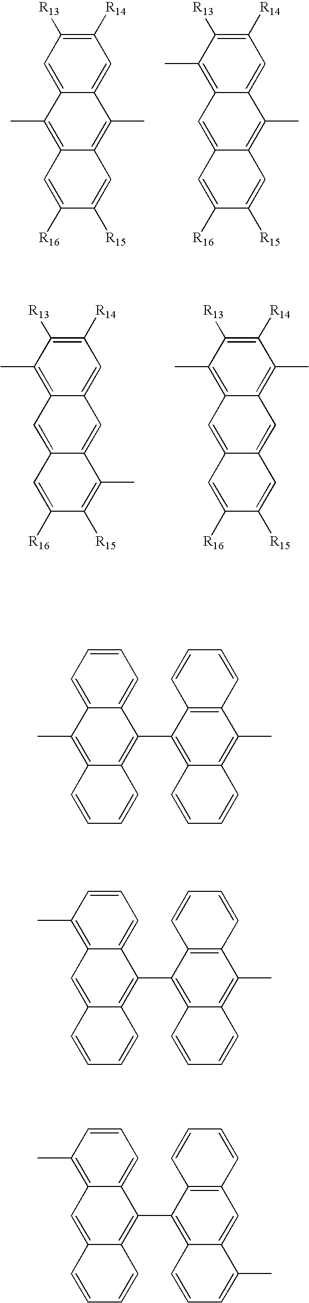 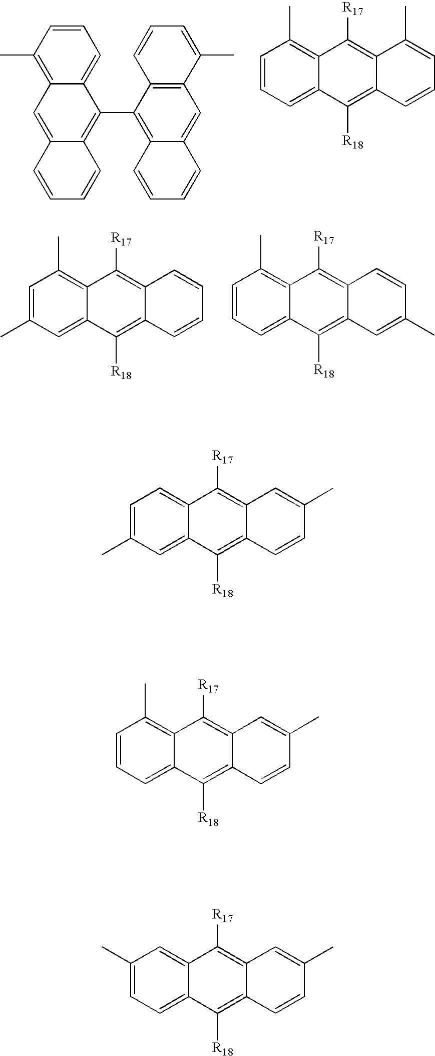
Ar₃ is selected from following structures; 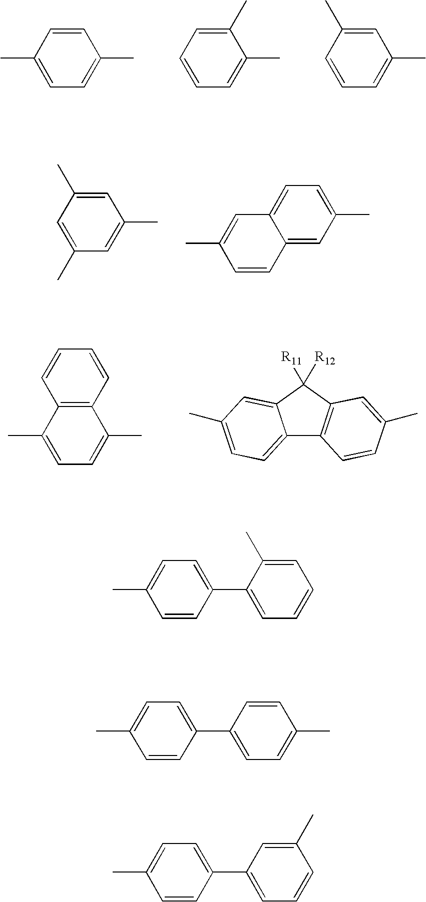
R₁ independently represents hydrogen, a C₁₋₂₀ alkyl group with or without halogen substituent(s), a C₁₋₂₀ alkylsilyl group, a C₆₋₂₀ arylsilyl group or a C₆₋₂₀ aryl group;
R₁₁ and R₁₂ independently represent hydrogen, a C₁₋₂₀ alkyl group with or without halogen substituent(s);
R₁₃ through R₁₈ independently represent hydrogen, a C₁₋₂₀ alkyl group with or without halogen substituent(s), a C₁₋₂₀ alkylsilyl group, a C₆₋₂₀ arylsilyl group or a C₆₋₂₀ aryl group;
n is 1 or 2; and
the aryl group of R₁ and R₁₃ through R₁₈ may further comprise C₁₋₂₀ alkyl group(s) or halogen substituent(s).

2. A thiazole system organic electroluminescent compound according to claim 1, which is selected from the compounds represented by one of Chemical Formulas (2) to (4): 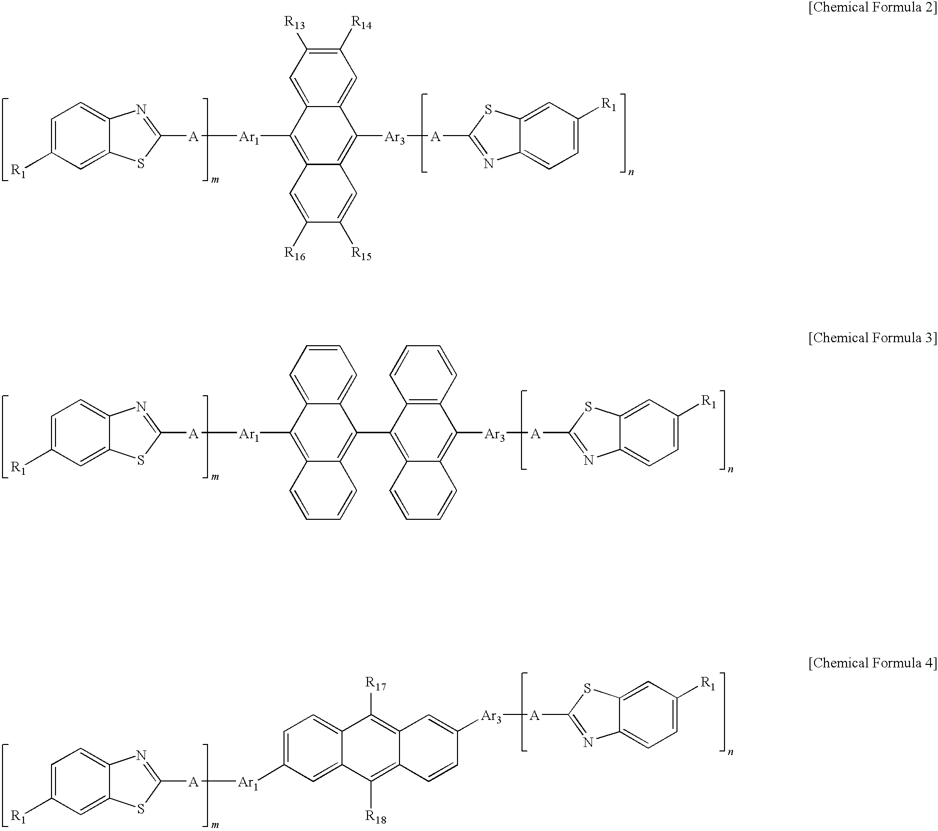
wherein, A, Ar₁, Ar₃, R₁, R₁₃, R₁₄, R₁₅, R₁₆, R₁₇, R₁₈, m and n are defined for Chemical Formula (1) in claim 1.

3. A thiazole system organic electroluminescent compound according to claim 2, wherein R₁ and R₁₃ through R₁₈ are independently selected from hydrogen, methyl, ethyl, n-propyl, i-propyl, i-butyl, t-butyl, n-pentyl, i-amyl, n-hexyl, n-heptyl, n-octyl, 2-ethylhexyl, n-nonyl, decyl, dodecyl, hexadecyl, trifluoromethyl, pentafluoroethyl, trimethylsilyl, tripropylsilyl, tri(t-buyl)silyl, t-butyldimethylsilyl, triphenylsilyl, phenyldimethylsilyl, phenyl, benzyl, tolyl, 2-fluorophenyl, 4-fluorophenyl, biphenyl, naphthyl, anthryl, phenanthryl, naphthacenyl, fluorenyl, 9,9-dimethylfluoren-2-yl, pyrenyl, phenylenyl and fluoranthenyl.

4. A thiazole system organic electroluminescent compound according to claim 3, which is selected from following compounds:

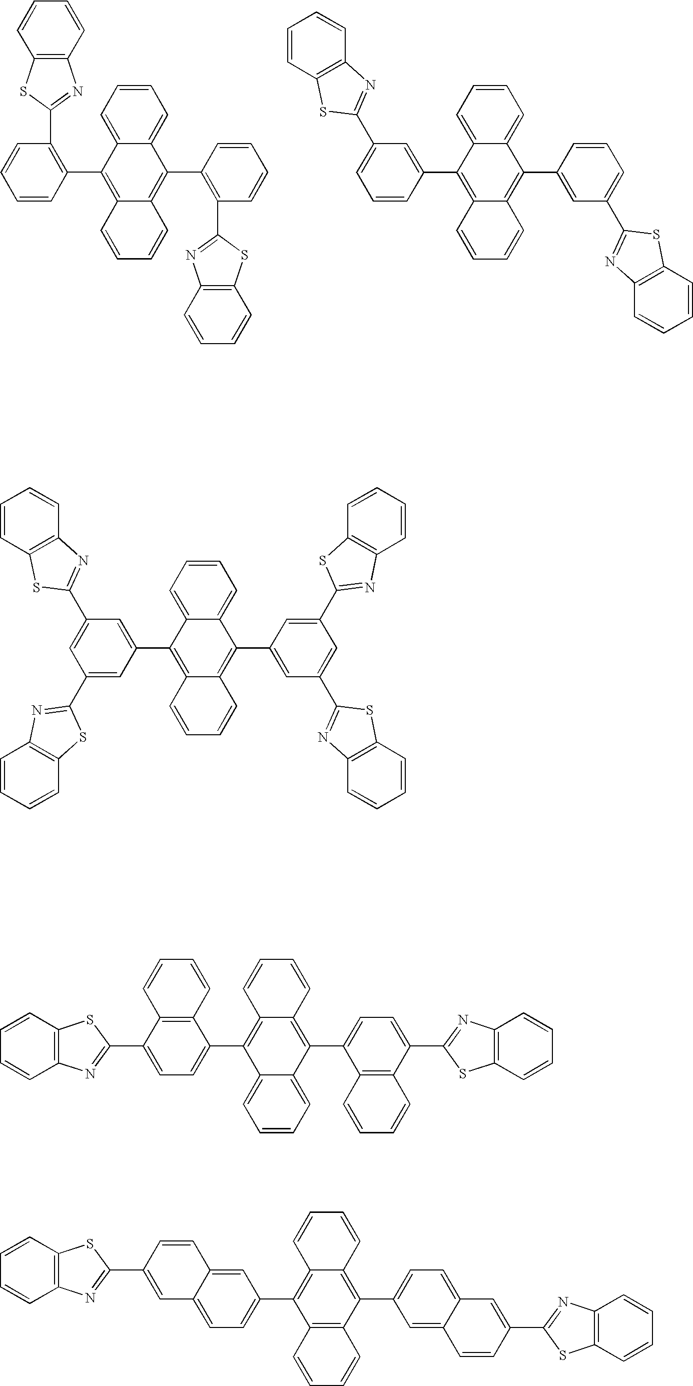

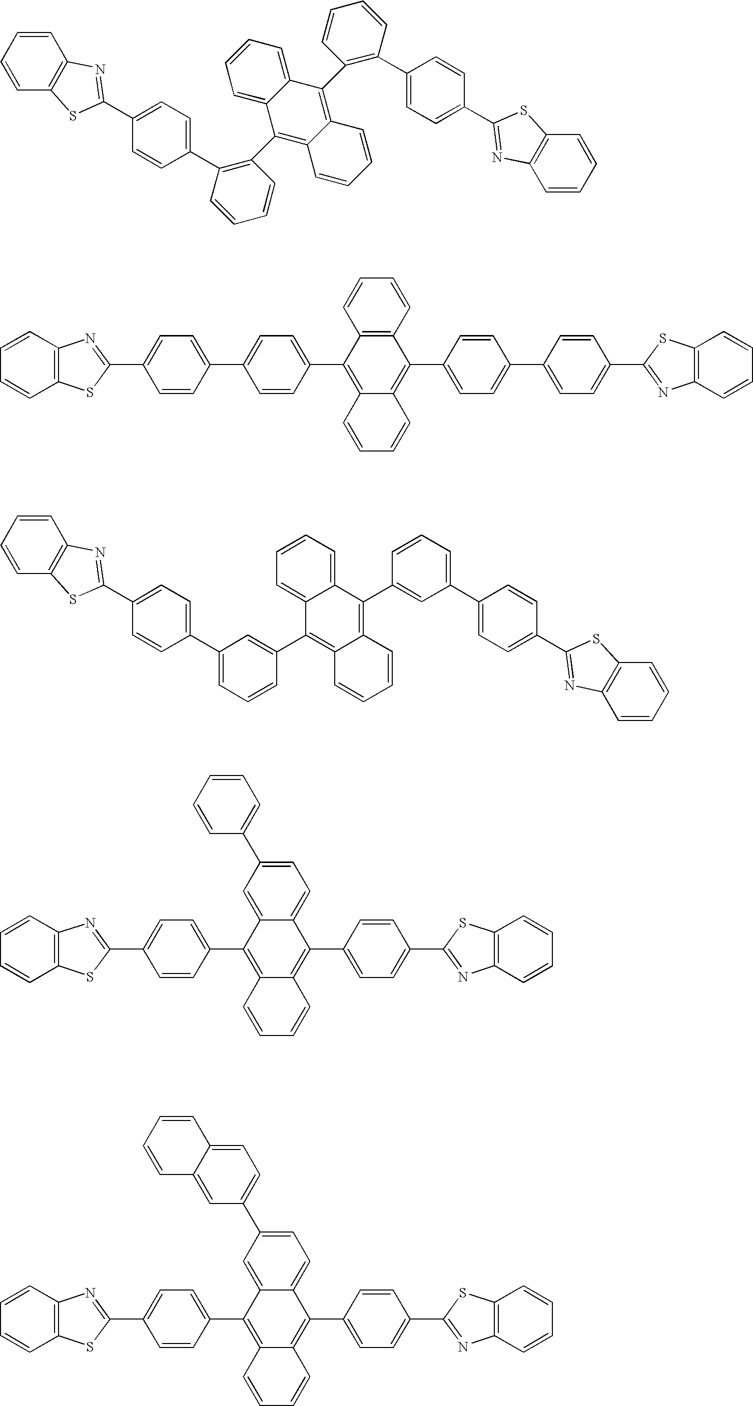

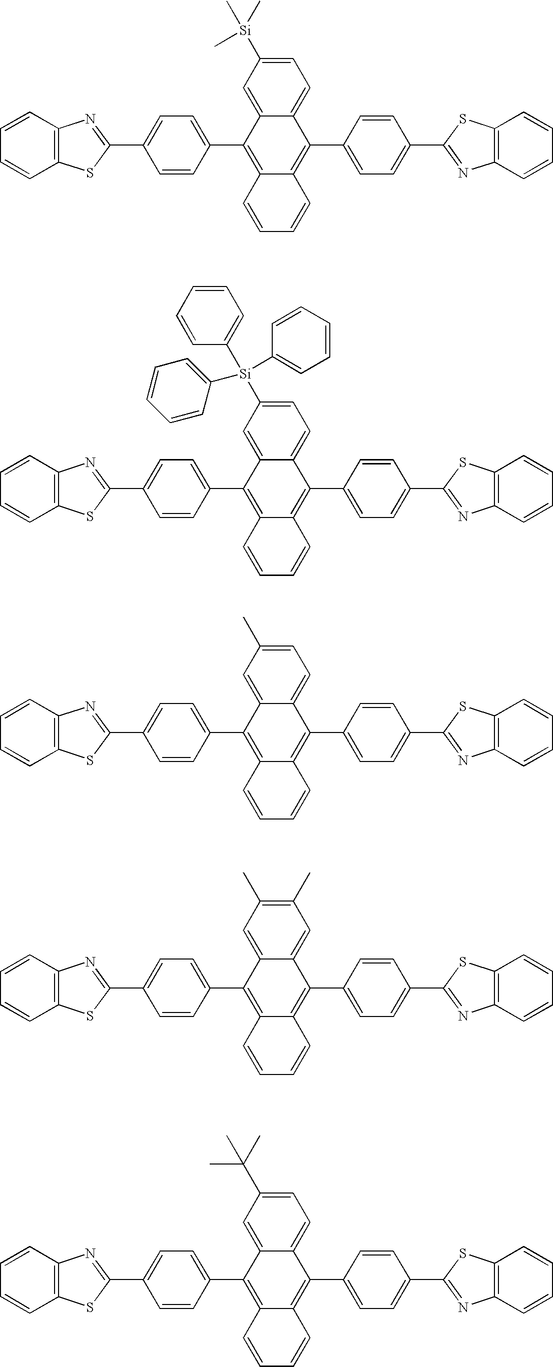

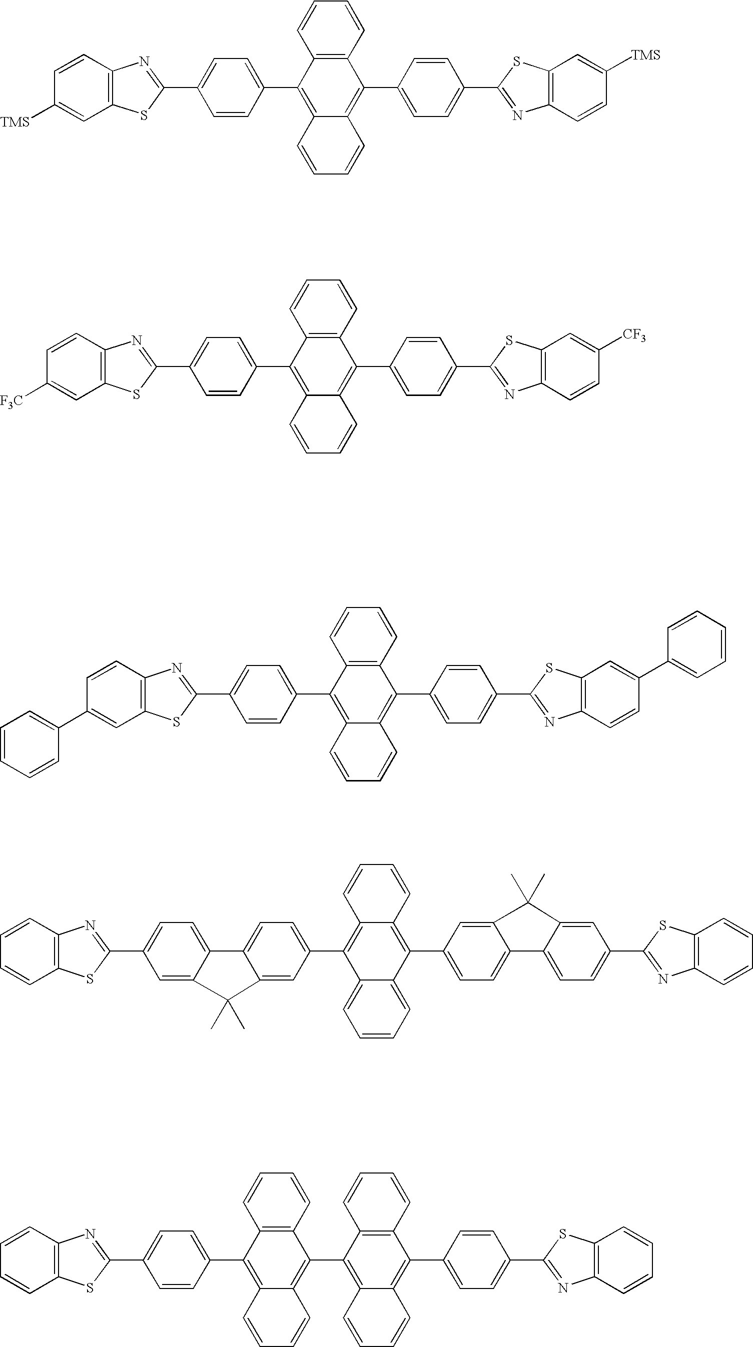

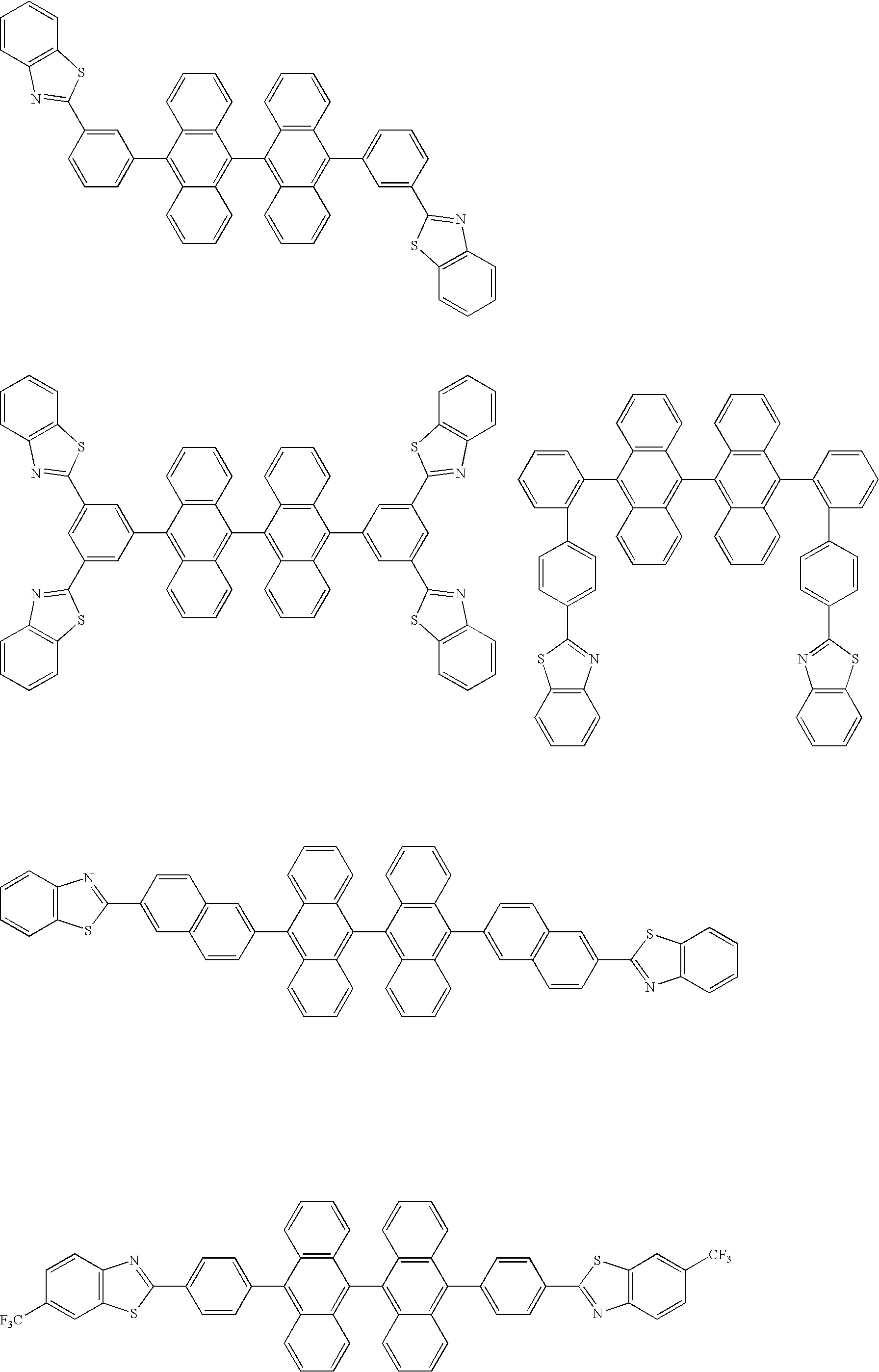

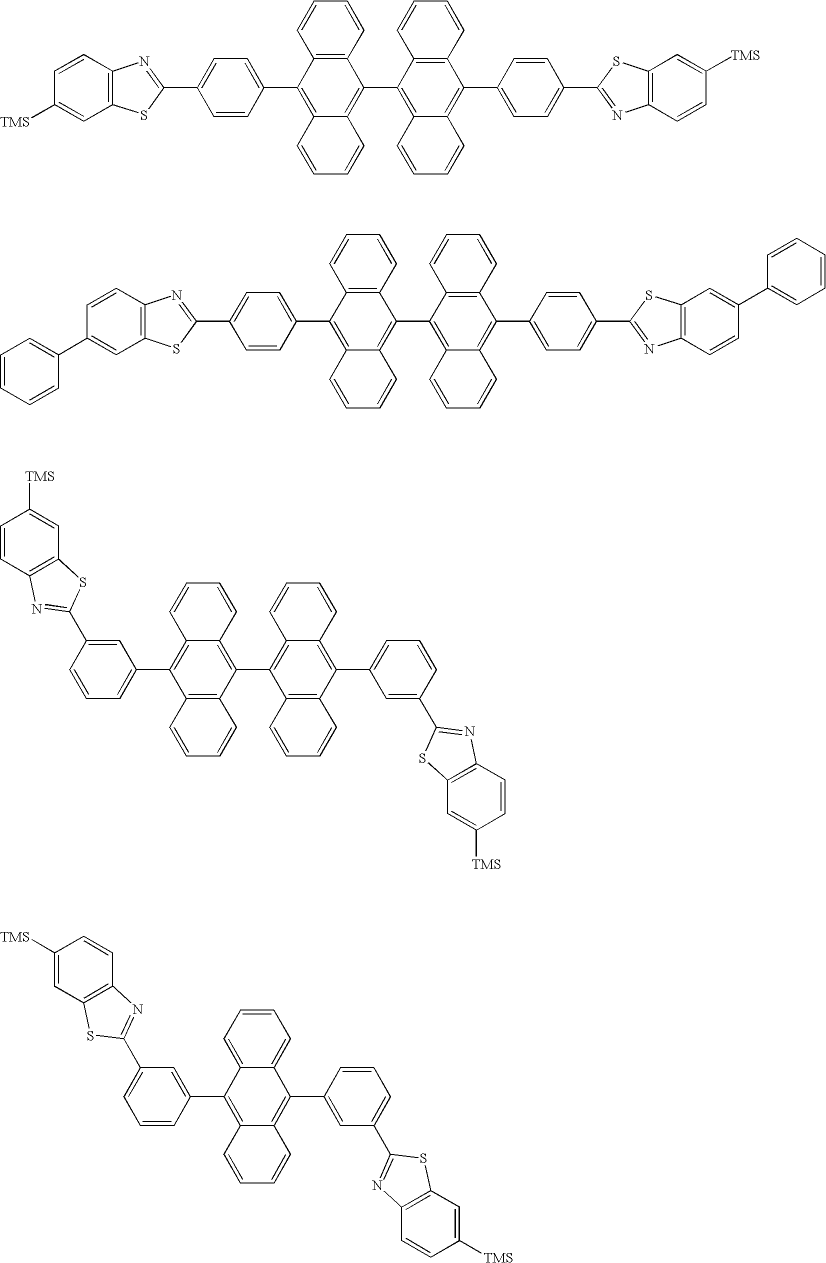

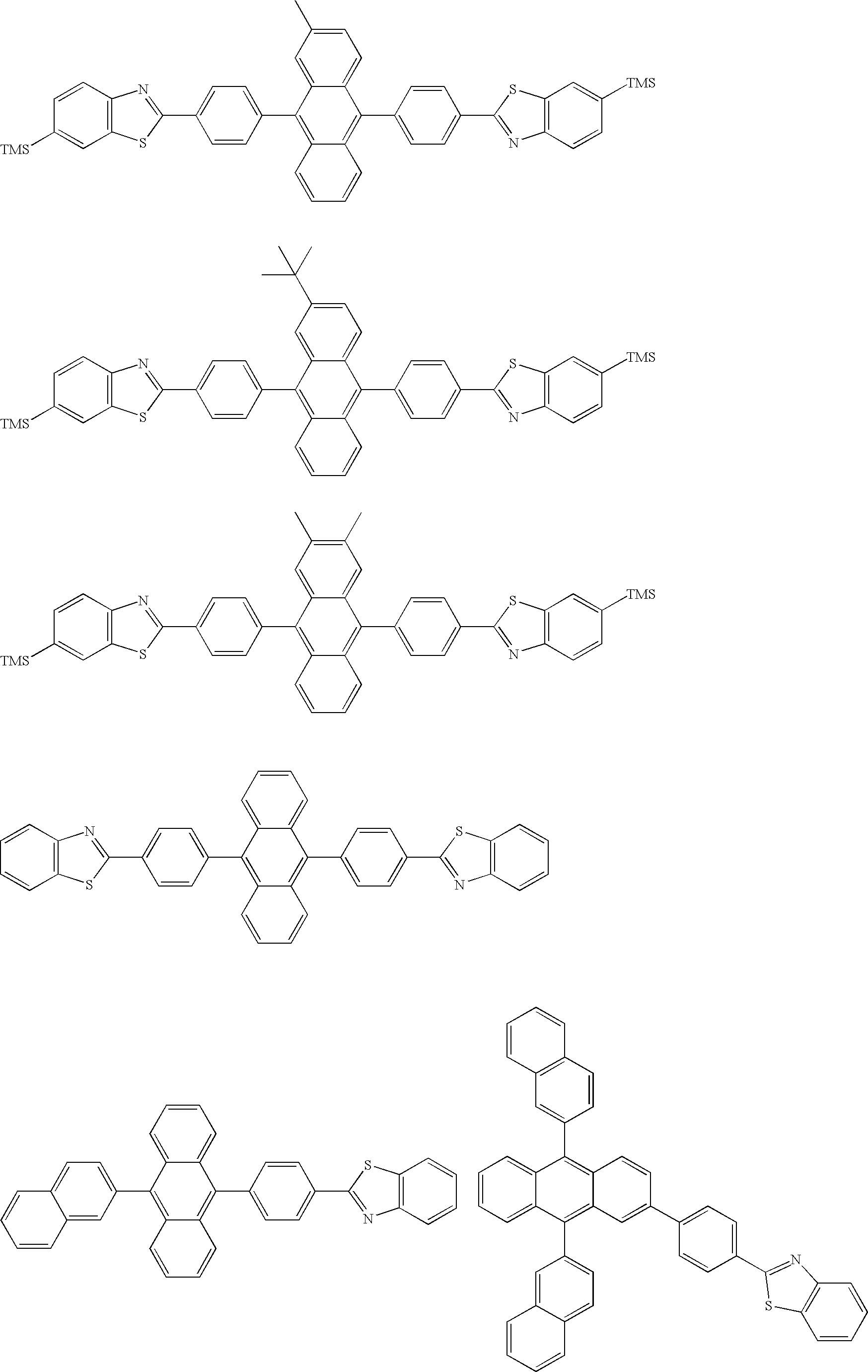

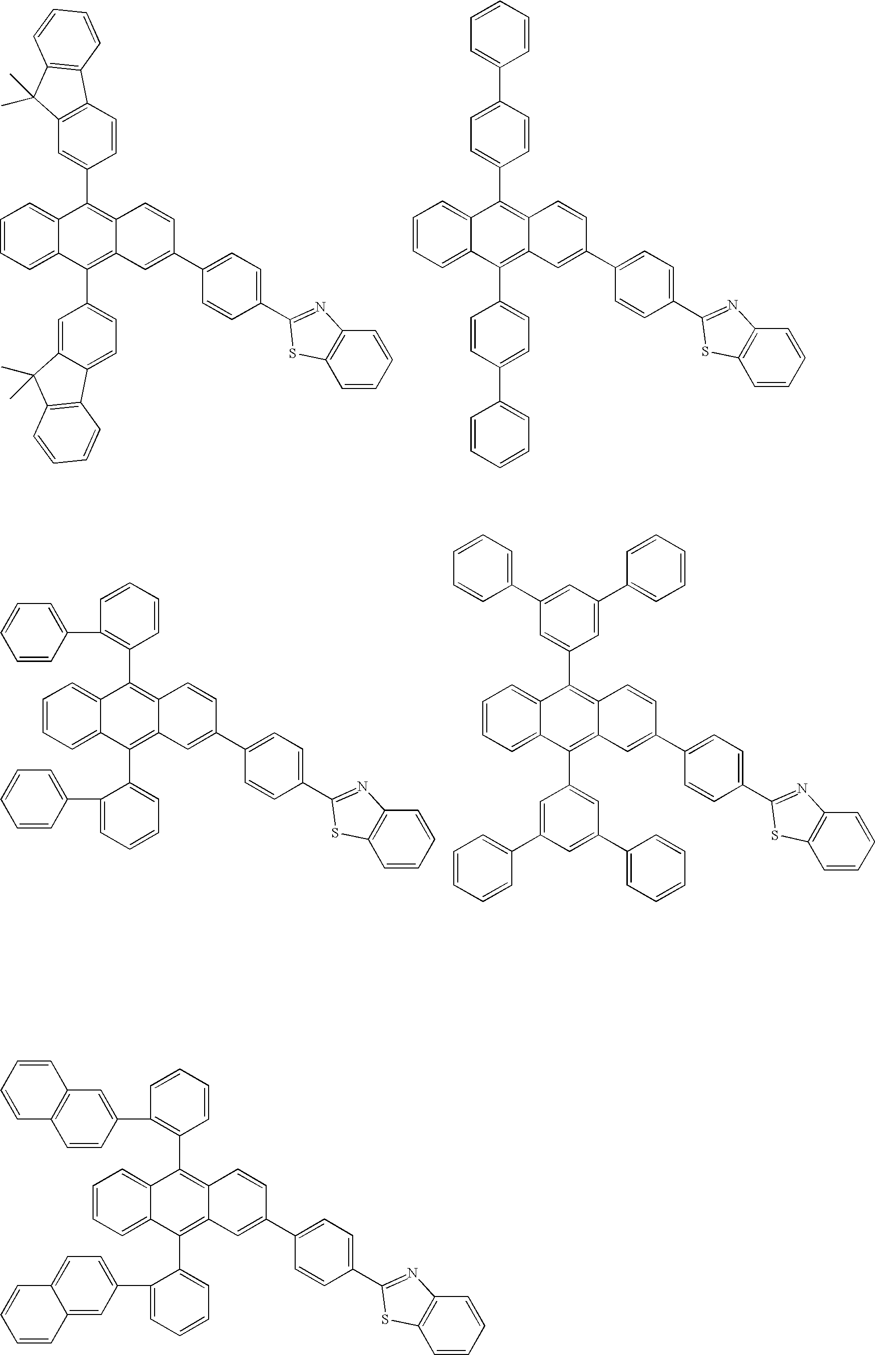

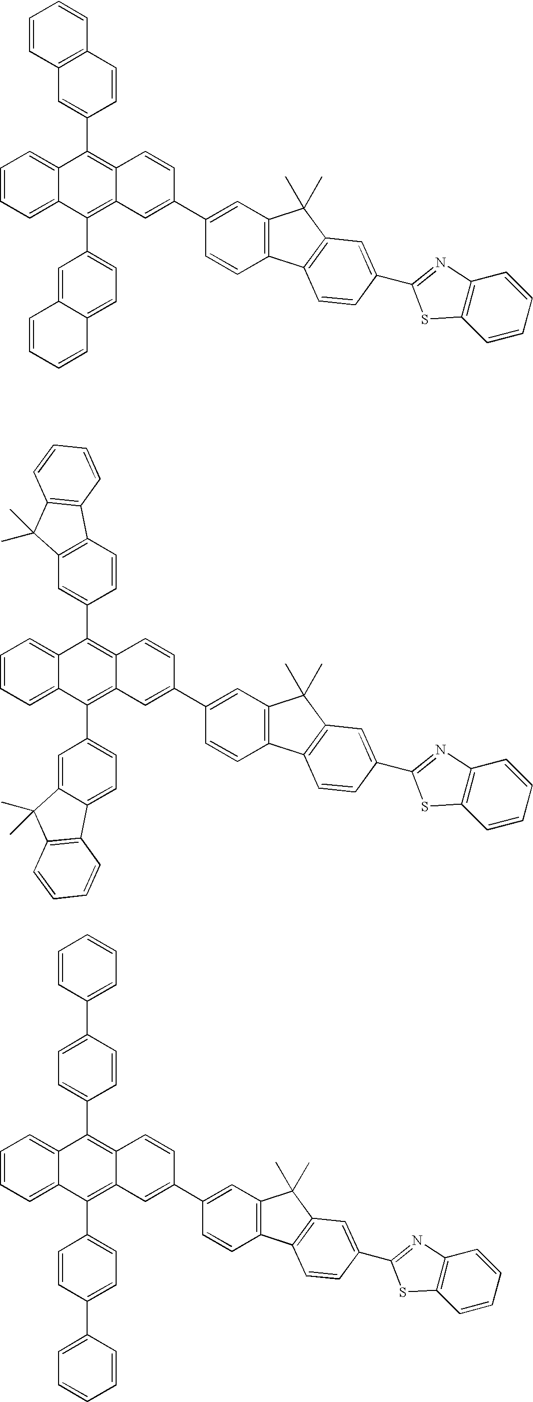

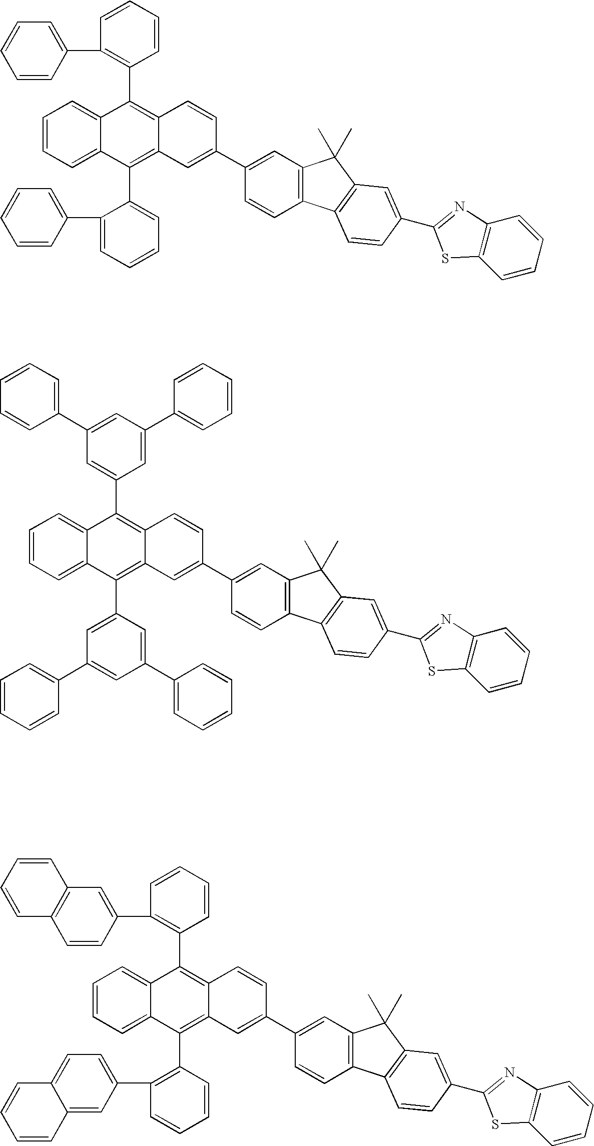

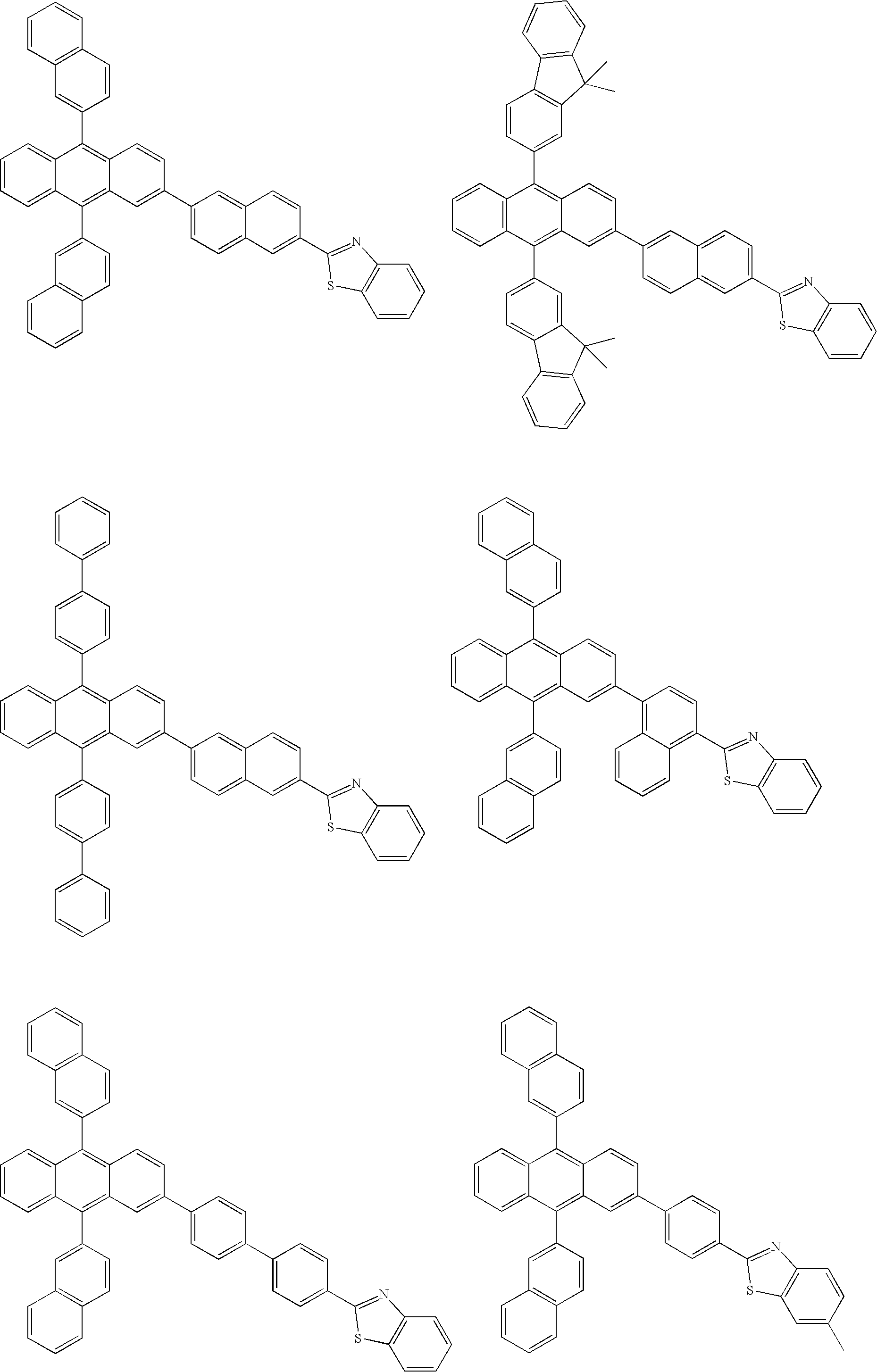

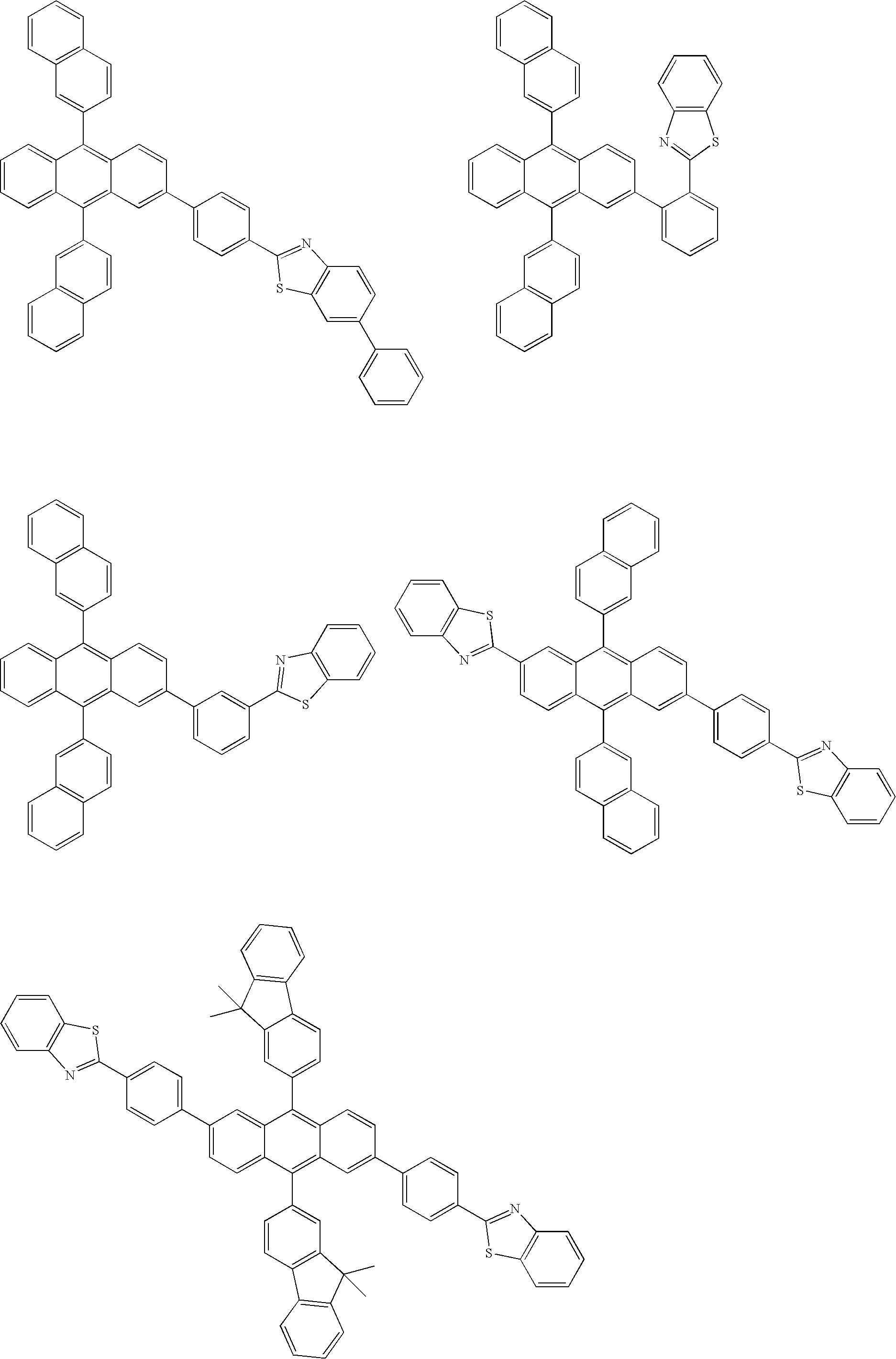

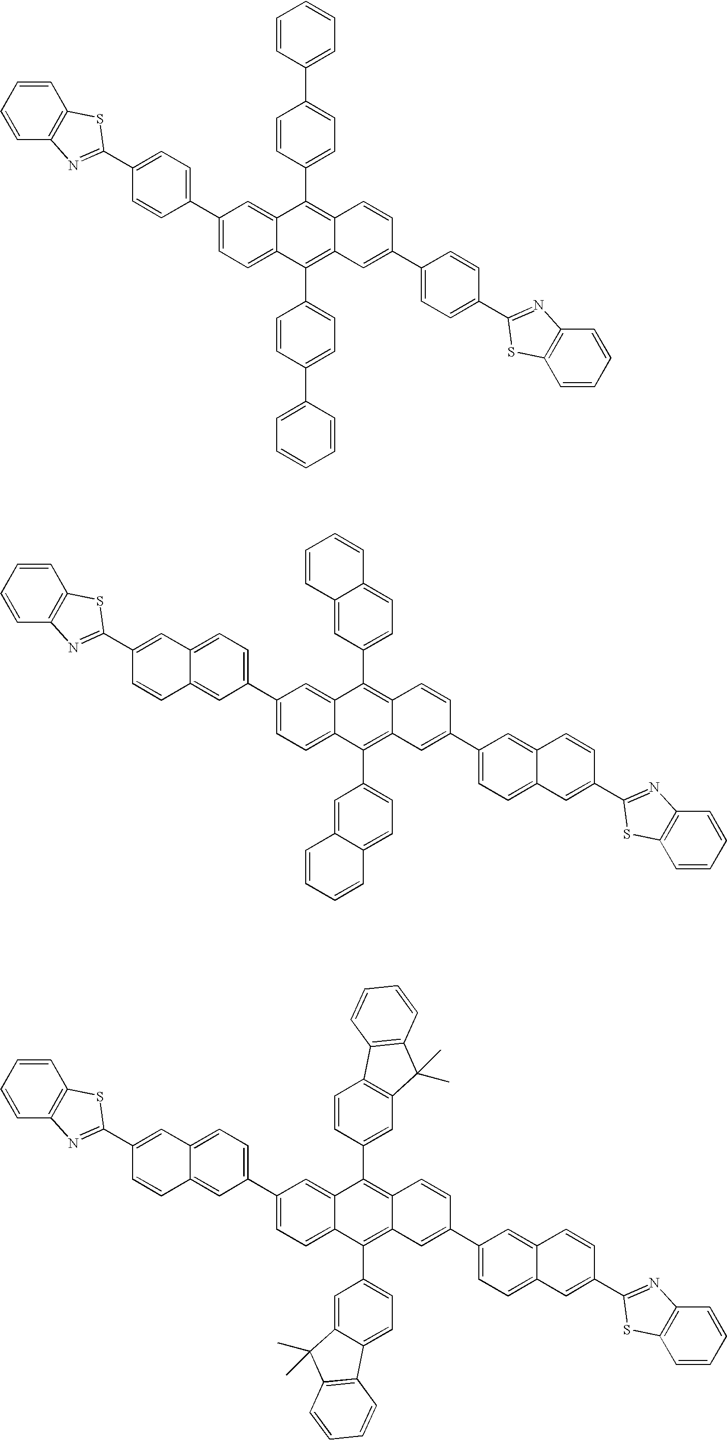

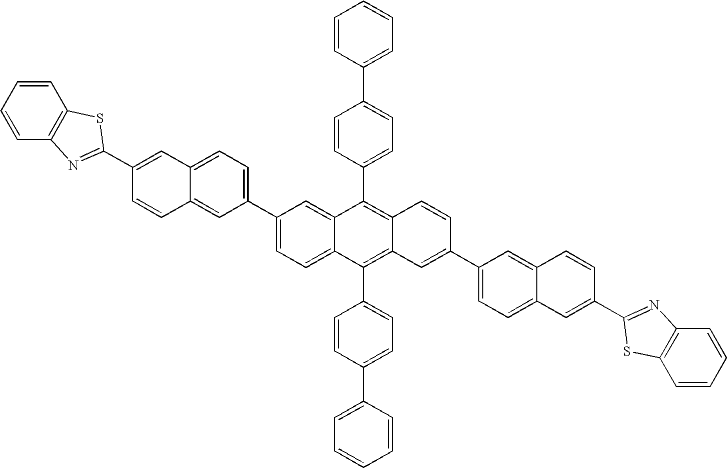

5. An organic light emitting diode comprising a thiazole system organic electroluminescent compound according to any one of claims 1 to 4.
